(19) United States
(12) Patent Application Publication
(10) Pub. No.: US 2024/0109883 A1
(43) Pub. Date: Apr. 4, 2024

(54) Anti-Tumor Compositions and Methods

(71) Applicant: Evrys Bio, LLC, Doylestown, PA (US)

(72) Inventors: STACY REMISZEWSKI, Township of Washington, NJ (US); LILLIAN W. CHIANG, Princeton, NJ (US); FRANK KAYSER, San Francisco, CA (US); SARAH JOCELYN FINK, Arlington, MA (US)

(73) Assignee: Evrys Bio, LLC, Doylestown, PA (US)

(21) Appl. No.: 18/270,288

(22) PCT Filed: Dec. 30, 2021
(86) PCT No.: PCT/US2021/065629

§ 371 Date: Jun. 29, 2023

Related U.S. Application Data
(60) Provisional application No. 63/132,798, filed on Dec. 31, 2020.

(51) Int. Cl. C07D 417/14; A61P 31/14; A61P 31/22; A61P 35/00; C07D 413/14; C07D 417/04; C07D 471/04

(52) U.S. Cl. CPC C07D 417/14; A61P 31/14; A61P 31/22; A61P 35/00; C07D 413/14; C07D 417/04; C07D 471/04

(57) ABSTRACT

Novel thiazole- and/or isoquinoline-containing compounds are presented that are useful for treating cancers as well as treating and/or preventing viral infections. Methods of treating and/or preventing broad-spectrum viral infections are also presented. These compounds have been shown to inhibit HCMV, influenza virus, and coronavirus replication in cell-based assays.

### STATEMENT OF FEDERALLY FUNDED RESEARCH

This invention was made with government support under grant numbers R44AI122488 and R44AI114079 awarded by the National Institutes of Health. The government has certain rights to the invention.

### TECHNICAL FIELD

This document relates to compounds useful for selectively killing tumor cells, and treating cell proliferative disorders.

### BACKGROUND

Unregulated cell growth is the hallmark of tumors and cancers and other cell proliferative disorders. The cellular processes controlling cell division and cell proliferation are complex, involving an intricate interplay between gene products that promote cell division and growth and those that hold such processes in check.

The TP53 gene, which encodes the p53 protein, is the most frequent target for mutation in tumors, with over half of all human cancers exhibiting mutation at this locus. Wild-type p53 functions primarily as a transcription factor and possesses an N-terminal transactivation domain, a centrally located sequence specific DNA binding domain, followed by a tetramerization domain and a C-terminal regulatory domain. Deficient p53 function has been shown to predict poor outcomes in multiple types of human tumors, including breast cancer, and certain mutants of p53 associate with an even worse prognosis. In response to a number of stressors, including DNA damage, hypoxia and oncogenic activation, p53 becomes activated to promote cell cycle arrest, apoptosis or senescence thereby suppressing tumor growth. It also plays many additional roles including regulating cellular metabolism. Enhanced activation of p53 in response to oncogenic stress is considered a promising anti-cancer mechanism.

The proto-oncoprotein Myc family is comprised of three members, c-Myc, N-Myc and L-myc. They are transcription factors that activate many growth promoting signal transduction pathways. Myc family members are often constitutively over-expressed in tumor cells, leading to the increased expression of many genes, some of which are involved in cell proliferation, contributing to the formation of cancer. More specifically, constitutive upregulation of c-Myc has been observed in carcinoma of the cervix, colon, breast, lung and stomach. c-Myc degradation or inactivation is thus viewed as a promising mechanism for anti-cancer drugs.

Aurora Kinase A is a member of a family of mitotic serine/threonine kinases. It is implicated in important processes during mitosis and meiosis whose proper function is integral for healthy cell proliferation. Aurora Kinase A disregulation is associated with multiple cancers. For example, one study showed over-expression of Aurora Kinase A in 94 percent of the invasive tissue growth in breast cancer, while surrounding, healthy tissues had normal levels of Aurora Kinase A expression. Degradation or inactivation of the kinase is thus viewed as a promising mechanism for anti-cancer drugs.

The AKT oncoprotein is associated with tumor cell survival, proliferation, and invasiveness. The activation of AKT is also one of the most frequent alterations observed in human tumor cells. Tumor cells that have constantly active AKT may depend on AKT for survival. Because of these AKT functions, AKT inhibitors may treat cancers such as neuroblastoma. Some Akt inhibitors have undergone clinical trials.

Human cytomegalovirus (HCMV) is a major cause of birth defects and opportunistic infections in immunosuppressed individuals, and a possible cofactor in certain cancers. Organ transplant patients under immunosuppressive therapy are at high risk for viral infections; activation of a latent virus as well as donor or community acquired primary infections can cause significant complications including graft rejection, morbidity, and mortality. Herpesviruses (e.g. HCMV, HSV-1), polyomaviruses (e.g. BKV and JCV), hepatitis viruses (HBV and HCV) and respiratory viruses (e.g. influenza A, adenovirus) are the 4 major viral classes infecting these patients. Cytomegalovirus (HCMV) is the most prevalent post-transplant pathogen; HCMV can infect most organs, and despite the availability of HCMV antivirals such as ganciclovir, nephrotoxic side effects and increasing rates of drug-resistance significantly reduce graft and patient survival. Evrys Bio, LLC, under their former name, FORGE Life Science, LLC, has previously disclosed thiazole-containing compounds which are active against HCMV replication in published patent applications WO 2016/077232, WO 2016/077240 and WO 2019/079519.

### SUMMARY

The invention provides compounds having the structure of Formula I:

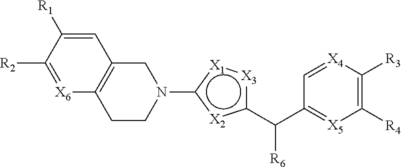

wherein:
one of X1, X2 and X3 is —S— or —O—, and
   X1, when not —S— or —O—, is —N—,
   X2, when not —S— or —O—, is —N—, and
   X3, when not —S— or —O—, is —N— or —C(R5)—, wherein R5 is selected from the group consisting of H, methyl, ethyl, n-propyl, isopropyl, n-butyl, CF₃, CH₂CF₃ and halo;
X4 is selected from —C(R13)— and —N—, wherein R13 is selected from the group consisting of H, methyl, ethyl, i-propyl or n-propyl;
X5 is selected from —CH— and —N—;
X6 is selected from —C(R14)— and —N—, wherein R14 is H or halo;
one of R1 and R2 is H and the other is

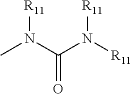

wherein R11 is independently selected, in each case, from H, methyl, ethyl, n-propyl & i-propyl;
R3 and R4 are independently selected from H, halo, —C≡CH, —C≡N, —OH, —OCF₃, —OCHF₂, C₁₋₄ straight or branched alkoxy optionally substituted with a 3- or 4-membered cycloalkyl, —SO₂(C₁₋₆alkyl), —N(CH₃)₂, —C(O)NH₂, —NHSO₂R7, —C(O)NR7R8, and a ring structure selected from a 5- or 6-membered aryl, a 3-, 4-, 5-, or 6-membered cylcloalkyl and a 3-, 4-, 5-, or 6-membered cycloalkoxy;
   wherein:
      each 5- or 6-membered aryl, 4-, 5-, or 6-membered cycloalkyl or 4-, 5-, or 6-membered cycloalkoxy has 0 to 3 ring heteroatoms and each 3-membered cycloalkyl or cycloalkoxy has 0 to 1 heteroatoms,
   each heteroatom is independently selected from N, O and S and
      each aryl, cycloalkyl or cycloalkoxy is substituted with 0 to 2 groups independently selected from:
         —O, —OH, halo, C₁₋₆ straight or branched alkyl optionally substituted with —OR12 or —NR7R8, C₁₋₆ straight or branched alkoxy optionally substituted with —NR7R8 or —OR12, —C(O)—C₁₋₆ alkyl and —C(O)O—C₁₋₆ alkyl, when the group is bonded to a carbon ring atom;
         —O⁻, C₁₋₆ straight or branched alkyl optionally substituted with —OR12 or —NR7R8, C₁₋₆ straight or branched alkoxy optionally substituted with —NR7R8 or —OR12, —C(O)—C₁₋₆ alkyl and —C(O)O—C₁₋₆ alkyl, when the group is bonded to a nitrogen ring atom; and
         ═O and (═O)₂ when the group is bonded to a sulfur ring atom;
      provided that, when —O⁻ is bonded to a nitrogen ring atom, the bond is

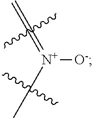

provided that:
   at least one of R3 and R4 is selected from the group consisting of: H, halo, —C≡CH, —C≡N, —OH, —OCF₃, —OCHF₂, C₁₋₄ straight or branched alkoxy optionally substituted with cyclopropyl or cyclobutyl, —SO₂(C₁₋₆ alkyl), —N(CH₃)₂, —C(O)NH₂, —NHSO₂R₇, and —C(O)NR7R8,
   when R3 or R4 is an aryl group, said aryl group is not substituted with ═O, and R3 and R4 are not both H;
R6 is selected from the group consisting of H, methyl, ethyl, n-propyl, isopropyl, n-butyl, CF₃, CH₂CF₃, halo, cyclopropylmethyl and C₁₋₄ alkoxy;
R7 and R8 are independently selected, in each instance, from H, C₁₋₆ straight or branched alkyl, C₃₋₆ cycloalkyl, cyclopropylmethyl and cyclobutylmethyl; and
R12 is independently selected, in each instance, from H and C₁₋₄ straight or branched alkyl;
or a pharmaceutically acceptable salt or solvate thereof.

The compounds of the invention are useful for selectively killing tumor cells and therefore treating cell-proliferative disorders and cancers. Compounds of the invention direct degradation of c-Myc oncoprotein in MDA-MB-231 triple-negative breast cancer cells, but not in “normal”, diploid MRC-5 fibroblasts. Compounds of the invention are Sirtuin 2 (SIRT2) inhibitors which stimulate the degradation of c-Myc oncoprotein and Aurora kinase A, activate p53 and prevent the full activation of AKT in tumor cells. As a consequence, they kill or stop the proliferation of tumor cells, including the transformed breast cancer cell line, MCF-7 cells. However, they do not inhibit the growth of non-transformed primary MRC-5 fibroblasts.

The invention also provides methods of treating and/or ameliorating viral infections, particularly HCMV, coronavirus or influenza infections with compounds of Formula I. The compounds of Formula I are broad-spectrum antiviral compounds.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF THE DRAWINGS

FIG. 1 presents the growth inhibition curve of MCF7 cells incubated with DMSO and four different concentrations of Example 003.

FIG. 2 presents the growth inhibition curve of MCF7 cells incubated with DMSO and four different concentrations of Example 004.

FIG. 3 presents the growth inhibition curve of MCF7 cells incubated with DMSO and four different concentrations of Example 001.

FIG. 4 presents the growth inhibition curve of MCF7 cells incubated with DMSO and four different concentrations of Example 024.

FIG. 5 presents the growth inhibition curve of MCF7 cells incubated with DMSO and four different concentrations of Example 023.

FIG. 6 presents the growth inhibition curve of MCF7 cells incubated with DMSO and four different concentrations of Example 021.

FIG. 7 presents the growth inhibition curve of MCF7 cells incubated with DMSO and four different concentrations of Example 020.

FIG. 8 presents the growth inhibition curve of MCF7 cells incubated with DMSO and four different concentrations of Example 019.

### DETAILED DESCRIPTION

Provided herein are compounds useful for selectively killing tumor cells and the treatment of cancers.

Provided herein are methods for treating a cell-proliferative disorder and/or cancer in a subject. In some embodiments, the methods include administering a therapeutically effective amount of one or more of the compounds provided herein. In some embodiments, the compounds provided herein can selectively kill tumor cells in a subject. In such embodiments, the subject is treated with a tumor cell killing amount of one or more compounds provided herein.

Provided herein are compounds of Formula I:

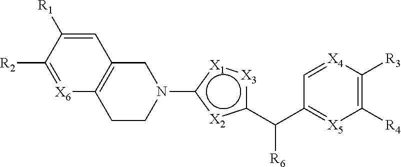

wherein:
one of X1, X2 and X3 is —S— or —O—, and
   X1, when not —S— or —O—, is —N—,
   X2, when not —S— or —O—, is —N—, and
   X3, when not —S— or —O—, is —N— or —C(R5)—, wherein R5 is selected from the group consisting of H, methyl, ethyl, n-propyl, isopropyl, n-butyl, CF₃, CH₂CF₃ and halo;
X4 is selected from —C(R13)— and —N—, wherein R13 is selected from the group consisting of H, methyl, ethyl, i-propyl or n-propyl;
X5 is selected from —CH— and —N—;
X6 is selected from —C(R14)— and —N—, wherein R14 is H or halo;
one of R1 and R2 is H and the other is

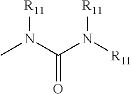

wherein R11 is independently selected, in each case, from H, methyl, ethyl, n-propyl & i-propyl;
R3 and R4 are independently selected from H, halo, —C≡CH, —C≡N, —OH, —OCF₃, —OCHF₂, C₁₋₄ straight or branched alkoxy optionally substituted with a 3- or 4-membered cycloalkyl, —SO₂(C₁₋₆alkyl), —N(CH₃)₂, —C(O)NH₂, —NHSO₂R7, —C(O)NR7R8, and a ring structure selected from a 5- or 6-membered aryl, a 3-, 4-, 5-, or 6-membered cylcloalkyl and a 3-, 4-, 5-, or 6-membered cycloalkoxy;
   wherein:
      each 5- or 6-membered aryl, 4-, 5-, or 6-membered cycloalkyl or 4-, 5-, or 6-membered cycloalkoxy has 0 to 3 ring heteroatoms and each 3-membered cycloalkyl or cycloalkoxy has 0 to 1 heteroatoms,
   each heteroatom is independently selected from N, O and S and
      each aryl, cycloalkyl or cycloalkoxy is substituted with 0 to 2 groups independently selected from:
         ═O, —OH, halo, C₁₋₆ straight or branched alkyl optionally substituted with —OR12 or —NR7R8, C₁₋₆ straight or branched alkoxy optionally substituted with —NR7R8 or —OR12, —C(O)—C₁₋₆ alkyl and —C(O)O—C₁₋₆ alkyl, when the group is bonded to a carbon ring atom;
         —O⁻, C₁₋₆ straight or branched alkyl optionally substituted with —OR12 or —NR7R8, C₁₋₆ straight or branched alkoxy optionally substituted with —NR7R8 or —OR12, —C(O)—C₁₋₆ alkyl and —C(O)O—C₁₋₆ alkyl, when the group is bonded to a nitrogen ring atom; and
         ═O and (═O)₂ when the group is bonded to a sulfur ring atom;
      provided that, when —O⁻ is bonded to a nitrogen ring atom, the bond is

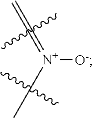

provided that:
   at least one of R3 and R4 is selected from the group consisting of: H, halo, —C≡CH, —C≡N, —OH, —OCF₃, —OCHF₂, C₁₋₄ straight or branched alkoxy optionally substituted with cyclopropyl or cyclobutyl, —SO₂(C₁₋₆ alkyl), —N(CH₃)₂, —C(O)NH₂, —NHSO₂R7, and —C(O)NR7R8,
   when R3 or R4 is an aryl group, said aryl group is not substituted with ═O, and R3 and R4 are not both H;
R6 is selected from the group consisting of H, methyl, ethyl, n-propyl, isopropyl, n-butyl, CF₃, CH₂CF₃, halo, cyclopropylmethyl and C₁₋₄ alkoxy;
R7 and R8 are independently selected, in each instance, from H, C₁₋₆ straight or branched alkyl, C₃₋₆ cycloalkyl, cyclopropylmethyl and cyclobutylmethyl; and
R12 is independently selected, in each instance, from H and C₁₋₄ straight or branched alkyl;
or a pharmaceutically acceptable salt or solvate thereof.
In some embodiments of the compounds of Formula I, one of R3 or R4 is:
a C₁₋₄ straight or branched alkoxy optionally substituted with a 3- or 4-membered cycloalkyl, or
a ring structure comprising a 5- or 6-membered aryl or a 3-, 4-, 5-, or 6-membered cylcloalkyl or a 3-, 4-, 5-, or 6-membered cycloalkoxy;
wherein:
   each 5- or 6-membered aryl, 4-, 5-, or 6-membered cycloalkyl or 4-, 5-, or 6-membered cycloalkoxy has 0 to 3 ring heteroatoms and each 3-membered cycloalkyl or cycloalkoxy has 0 to 1 heteroatoms;
   each heteroatom is independently selected from N, O and S; and
   each aryl, cycloalkyl or cycloalkoxy is substituted with 0 to 2 groups independently selected from:
      ═O, —OH, halo, C₁₋₆ straight or branched alkyl optionally substituted with —OR12 or —NR7R8, C₁₋₆ straight or branched alkoxy optionally substituted with —NR7R8 or —OR12, —C(O)—C₁₋₆ alkyl and —C(O)O—C₁₋₆ alkyl when bonded to a carbon ring atom;
      —O⁻, C₁₋₆ straight or branched alkyl optionally substituted with —OR12 or —NR7R8, C₁₋₆ straight or branched alkoxy optionally substituted with —NR7R8 or —OR12, —C(O)—C₁₋₆ alkyl and —C(O)O—C₁₋₆ alkyl when bonded to a nitrogen ring atom; and
      ═O and (═O)₂ when bonded to a sulfur ring atom
         provided that, when —O⁻ is bonded to a nitrogen ring atom, the bond is

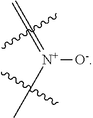

In some embodiments of the compounds of Formula I, wherein:
   one of R3 or R4 is:
      a C₁₋₄ straight or branched alkoxy optionally substituted with a 3- or 4-membered cycloalkyl, or
      a ring structure comprising a 5- or 6-membered aryl or a 3-, 4-, 5-, or 6-membered cylcloalkyl or a 3-, 4-, 5-, or 6-membered cycloalkoxy; and
   X2 is —S— or —O—.
In some embodiments of the compounds of Formula I, wherein:
   one of R3 or R4 is:
      a C₁₋₄ straight or branched alkoxy optionally substituted with a 3- or 4-membered cycloalkyl, or
      a ring structure comprising a 5- or 6-membered aryl or a 3-, 4-, 5-, or 6-membered cylcloalkyl or a 3-, 4-, 5-, or 6-membered cycloalkoxy;
   X2 is —S— or —O—; and
   X3 is —C(R5)—.
In some embodiments of the compounds of Formula I, wherein:
   one of R3 or R4 is:
      a C₁₋₄ straight or branched alkoxy optionally substituted with a 3- or 4-membered cycloalkyl, or
      a ring structure comprising a 5- or 6-membered aryl or a 3-, 4-, 5-, or 6-membered cylcloalkyl or a 3-, 4-, 5-, or 6-membered cycloalkoxy; and
   X2 is —S—.
In some embodiments of the compounds of Formula I, wherein:
   one of R3 or R4 is:
      a C₁₋₄ straight or branched alkoxy optionally substituted with a 3- or 4-membered cycloalkyl, or
      a ring structure comprising a 5- or 6-membered aryl or a 3-, 4-, 5-, or 6-membered cylcloalkyl or a 3-, 4-, 5-, or 6-membered cycloalkoxy;
   X2 is —S—; and
   X3 is —C(R5)—.
In some embodiments of the compounds of Formula I,
R3 is selected from the group consisting of:

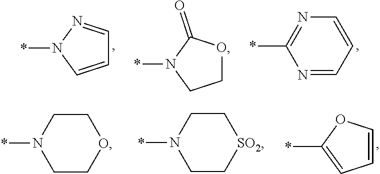

—SO₂(C₁₋₆ alkyl) and C₁₋₄ straight or branched alkoxy, wherein the alkoxy is optionally substituted with a 3- or 4-membered cycloalkyl,
   wherein:
   the 3- or 4-membered cycloalkyl has 0 to 1 heteroatoms independently selected from N, O and S; and
   the 3- or 4-membered cycloalkyl is substituted with 0 to 2 groups independently selected from:
      ═O, —OH, halo, C₁₋₄ straight or branched alkyl optionally substituted with —OR12 or —NR7R8, C₁₋₄ straight or branched alkoxy optionally substituted with —NR7R8 or —OR12, —C(O)—C₁₋₄ alkyl and —C(O)O—C₁₋₄ alkyl, when bonded to a carbon ring atom; and
      C₁₋₆ straight or branched alkyl optionally substituted with —OR12 or —NR7R8, C₁₋₆ straight or branched alkoxy optionally substituted with —NR7R8 or —OR12, —C(O)—C₁₋₄ alkyl and —C(O)O—C₁₋₄ alkyl, when bonded to a nitrogen ring atom.
In some of the embodiments of the compounds of Formula I, the compound is selected from the group consisting of:

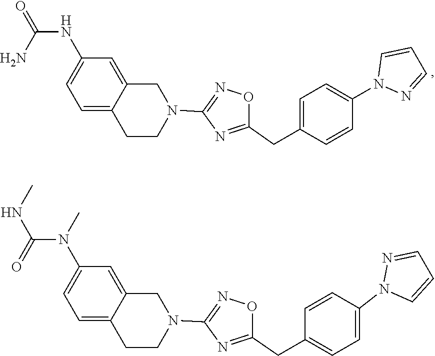 [SMILES: NC(=O)Nc1ccc2c(c1)CN(c1noc(Cc3ccc(-n4cccn4)cc3)n1)CC2 ; CNC(=O)N(C)c1ccc2c(c1)CN(c1noc(Cc3ccc(-n4cccn4)cc3)n1)CC2]

and a pharmaceutically acceptable solvate thereof.
In some embodiments of the compounds of Formula I, wherein:
R3 is selected from the group consisting of:

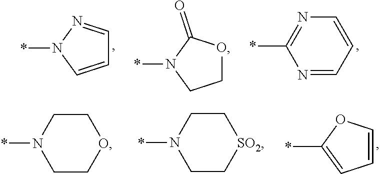

—SO₂(C₁₋₆ alkyl) and C₁₋₄ straight or branched alkoxy, wherein the alkoxy is optionally substituted with a 3- or 4-membered cycloalkyl; and
X2 is —S— or —O—.

In some of embodiments of the compounds of Formula I, the compound is selected from the group consisting of:

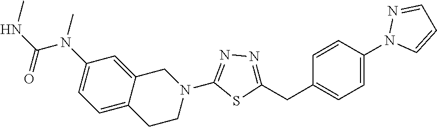 [SMILES: CNC(=O)N(C)c1ccc2c(c1)CN(c1nnc(Cc3ccc(-n4cccn4)cc3)s1)CC2]

and a pharmaceutically acceptable solvate thereof.
In some embodiments of the compounds of Formula I, wherein:
R3 is selected from the group consisting of:

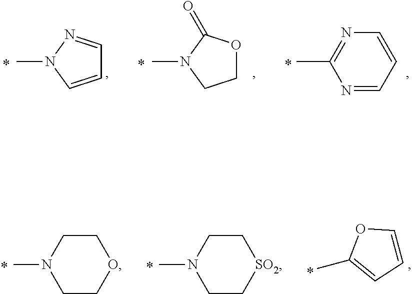

—SO₂(C₁₋₆ alkyl) and C₁₋₄ straight or branched alkoxy, wherein the alkoxy is optionally substituted with a 3- or 4-membered cycloalkyl;
X2 is —S— or —O—; and
X3 is —C(R5)—.
In some embodiments of the compounds of Formula I, wherein:
R3 is selected from the group consisting of:

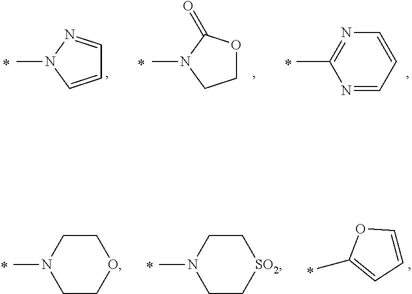

—SO₂(C₁₋₆ alkyl) and C₁₋₄ straight or branched alkoxy, wherein the alkoxy is optionally substituted with a 3- or 4-membered cycloalkyl; and
X2 is —S—.
In some embodiments of the compounds of Formula I, wherein:
R3 is selected from the group consisting of:

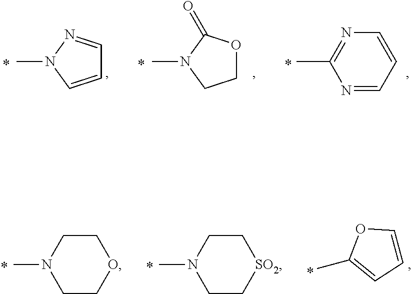

—SO₂(C₁₋₆ alkyl) and C₁₋₄ straight or branched alkoxy, wherein the alkoxy is optionally substituted with a 3- or 4-membered cycloalkyl;
X2 is —S—; and
X3 is —C(R5)—.
In some embodiments of the compounds of Formula I, the compound is selected from the group consisting of:

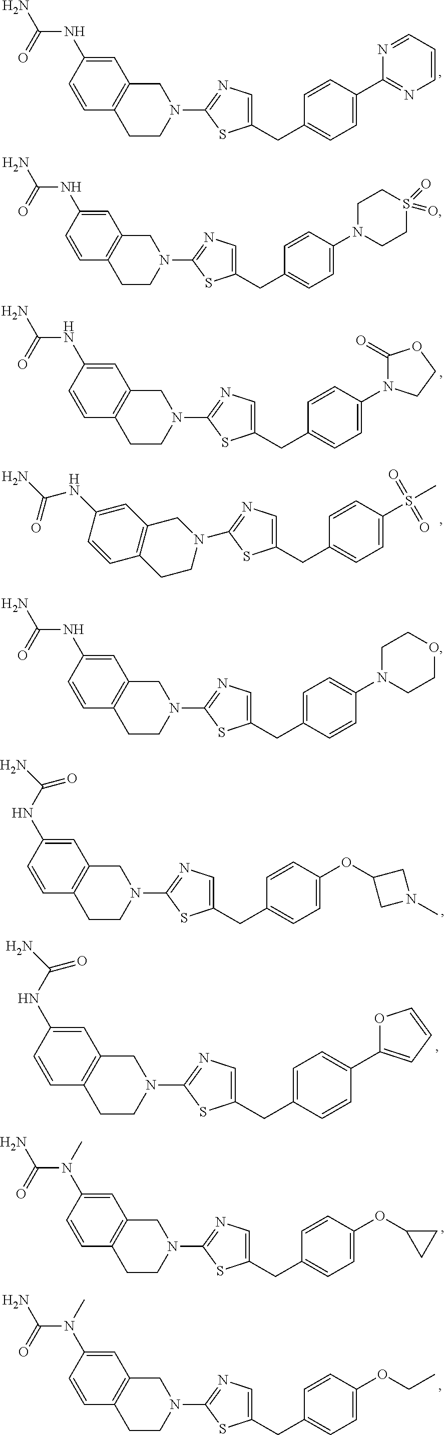

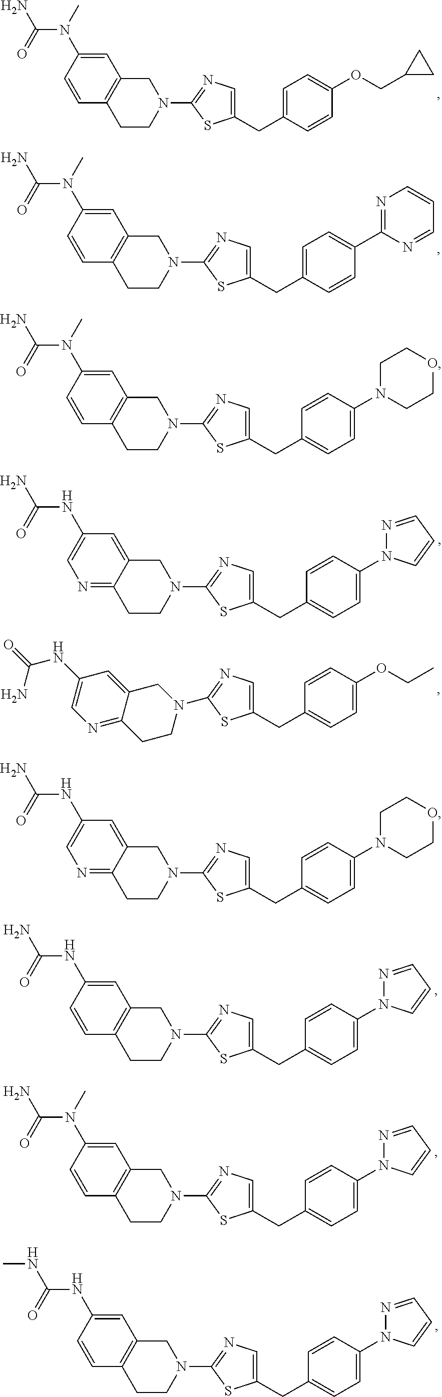

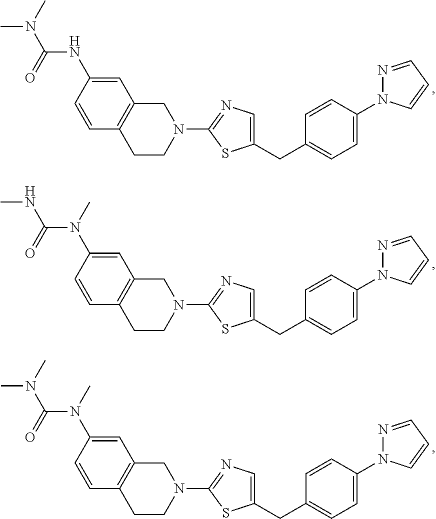 [SMILES: CN(C)C(=O)Nc1ccc2c(c1)CN(c1ncc(Cc3ccc(-n4cccn4)cc3)s1)CC2 ; CNC(=O)N(C)c1ccc2c(c1)CN(c1ncc(Cc3ccc(-n4cccn4)cc3)s1)CC2 ; CN(C)C(=O)N(C)c1ccc2c(c1)CN(c1ncc(Cc3ccc(-n4cccn4)cc3)s1)CC2]

and a pharmaceutically acceptable salt or solvate thereof.
In some embodiments of the compounds of Formula I,
R4 is selected from the group consisting of:

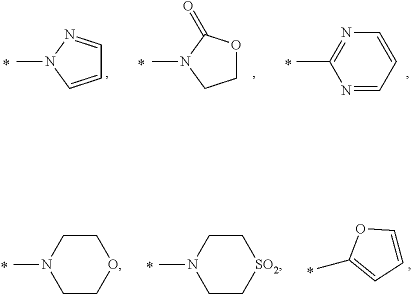

—SO₂(C₁₋₆ alkyl) and C₁₋₄ straight or branched alkoxy, wherein the alkoxy is optionally substituted with a 3- or 4-membered cycloalkyl,
   wherein:
   the 3- or 4-membered cycloalkyl has 0 to 1 heteroatoms independently selected from N, O and S; and
   the 3- or 4-membered cycloalkyl is substituted with 0 to 2 groups independently selected from:
      ═O, —OH, halo, C₁₋₄ straight or branched alkyl optionally substituted with —OR12 or —NR7R8, C₁₋₄ straight or branched alkoxy optionally substituted with —NR7R8 or —OR12, —C(O)—C₁₋₄ alkyl and —C(O)O—C₁₋₄ alkyl when bonded to a carbon ring atom; and
      C₁₋₆ straight or branched alkyl optionally substituted with —OR12 or —NR7R8, C₁₋₆ straight or branched alkoxy optionally substituted with —NR7R8 or —OR12, —C(O)—C₁₋₄ alkyl and —C(O)O—C₁₋₄ alkyl when bonded to a nitrogen ring atom.

Also provided herein is a pharmaceutical composition comprising a compound of Formula I or any of the above embodiments of Formula I and a pharmaceutically acceptable excipient.

Also provided herein is a method of treating cancer in a patient in need of treatment comprising administering to said patient a therapeutically effective amount of a compound of Formula I or any of the above embodiments of Formula I.

Also provided herein is a method of treating breast cancer in a patient in need of treatment comprising administering to said patient a therapeutically effective amount of a compound of Formula I or any of the above embodiments of Formula I.

Also provided herein is a method for treating or preventing a viral infection in a subject comprising administering a therapeutically effective amount of a compound of Formula I or pharmaceutically acceptable salts or solvates thereof.

Also provided herein is a method of inhibiting virus production comprising contacting a virus-infected cell with a virus production inhibiting amount of a compound of Formula I or pharmaceutically acceptable salts or solvates thereof.

Also provided herein is a method for treating or preventing an HCMV infection in a subject by administering a therapeutically effective amount of a compound of Formula I or pharmaceutically acceptable salts or solvates thereof.

Also provided herein is a method of inhibiting HCMV production comprising contacting an HCMV-infected cell with a virus production inhibiting amount of a compound of Formula I, or pharmaceutically acceptable salts or solvates thereof.

Also provided herein is a method of treating or preventing a coronavirus infection in a subject by administering a therapeutically effective amount of a compound of Formula I or pharmaceutically acceptable salts or solvates thereof.

Also provided herein is a method of inhibiting coronavirus production comprising contacting a coronavirus-infected cell with a virus production inhibiting amount of a compound of Formula I, or pharmaceutically acceptable salts or solvates thereof.

Also provided herein is a method for treating or preventing an influenza infection in a subject by administering a therapeutically effective amount of a compound of Formula I or pharmaceutically acceptable salts or solvates thereof.

Also provided herein is a method of inhibiting influenza A production comprising contacting an influenza A virus-infected cell with a virus production inhibiting amount of a compound of Formula I or pharmaceutically acceptable salts or solvates thereof.

An antiviral agent can also be administered in conjunction with the compounds and the methods described herein. The agent can be any therapeutic agent useful in the treatment of a viral infection, an HCMV infection or an influenza infection. For example, an antiviral agent can include acyclovir, docosanol, ribarivin, interferons, and the like; cellulose acetate, carbopol and carrageenan, pleconaril, amantidine, rimantidine, fomivirsen, zidovudine, lamivudine, zanamivir, oseltamivir, brivudine, abacavir, adefovir, amprenavir, arbidol, atazanavir, atripla, cidofovir, combivir, edoxudine, efavirenz, emtricitabine, enfuvirtide, entecavir, famciclovir, fosamprenavir, foscarnet, fosfonet, ganciclovir, gardasil, ibacitabine, imunovir, idoxuridine, imiquimod, indinavir, inosine, integrase inhibitor, lamivudine, lopinavir, loviride, mk-0518, maraviroc, moroxydine, nelfinavir, nevirapine, nexavir, nucleotide and/or nucleoside analogues, oseltamivir, penciclovir, peramivir, podophyllotoxin, rimantadine, ritonavir, saquinavir, stavudine, tenofovir, tenofovir disoproxil, tipranavir, trifluridine, trizivir, tromantadine, truvada, valaciclovir, valganciclovir, vicriviroc, vidarabine, viramidine, zalcitabine, morpholino oligonucleotides, ribozyme, protease inhibitors, an assembly inhibitor (e.g., rifampicin), zidovudine, brincidofovir, favipiravir, nitoxanide, letermovir, maribavir, CMX157 or a combination or two or more antiviral agents.

In some embodiments, a compound provided herein can be administered before, after, or simultaneously with the administration or one or more antiviral agents.

An antiviral agent provided herein, including a pharmaceutically acceptable salt or solvate thereof, can be purchased commercially or prepared using known organic synthesis techniques.

The methods provided herein include the manufacture and use of pharmaceutical compositions, which include compounds provided herein and one or more pharmaceutically acceptable carriers. Also provided herein are the compositions themselves.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration.

A pharmaceutical composition is typically formulated to be compatible with its intended route of administration. Examples of routes of administration include parenteral, e.g., intravenous, intradermal, subcutaneous, oral (e.g., inhalation), transdermal (topical), transmucosal, and rectal administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral, intradermal, or subcutaneous application can include the following components: a sterile diluent such as water for injection, saline solution, fixed oils, polyethylene glycols, glycerine, propylene glycol, or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates, or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes, or multiple dose vials made of glass or plastic.

Pharmaceutical compositions suitable for injection can include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL™ (BASF, Parsippany, NJ) or phosphate buffered saline (PBS). In all cases, the composition must be sterile and should be fluid to the extent that easy syringability exists. The composition should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions can be brought about by including in the composition an agent that delays absorption, for example, aluminum monostearate and gelatin.

Sterile injectable solutions can be prepared by incorporating a compound provided herein in the required amount in an appropriate solvent with one or a combination of ingredients enumerated above, as required, followed by filtered sterilization. Generally, dispersions are prepared by incorporating a compound provided herein into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of a compound provided herein plus any additional desired ingredient from a previously sterile-filtered solution thereof.

Oral compositions generally include an inert diluent or an edible carrier. For the purpose of oral therapeutic administration, a compound provided herein can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressured container or dispenser that contains a suitable propellant, e.g., a gas such as carbon dioxide, or a nebulizer. Such methods include those described in U.S. Pat. No. 6,468,798. Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the compounds provided herein can be formulated into ointments, salves, gels, or creams as generally known in the art.

The pharmaceutical compositions can also be prepared in the form of suppositories (e.g., with conventional suppository bases such as cocoa butter and other glycerides) or retention enemas for rectal delivery.

Additionally, intranasal delivery is possible, as described in, inter alia, Hamajima et al., Clin. Immunol. Immunopathol., 88(2), 205-10 (1998). Liposomes (e.g., as described in U.S. Pat. No. 6,472,375) and microencapsulation can also be used. Biodegradable targetable microparticle delivery systems can also be used (e.g., as described in U.S. Pat. No. 6,471,996).

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811.

The pharmaceutical composition may be administered at once or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular patient, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

Dosage forms or compositions containing a compound as described herein in the range of 0.005% to 100% with the balance made up from non-toxic carrier may be prepared. Methods for preparation of these compositions are known to those skilled in the art. The contemplated compositions may contain 0.001%-100% of a compound provided herein, in one embodiment 0.1-95%, in another embodiment 75-85%.

The pharmaceutical compositions can be included in a container, pack, or dispenser together with instructions for administration.

As described above, the preparations of one or more compounds provided herein may be given orally, parenterally, topically, or rectally. They are, of course, given by forms suitable for each administration route. For example, they are administered in tablets or capsule form, by injection, inhalation, eye lotion, ointment, suppository, infusion; topically by lotion or ointment; and rectally by suppositories. In some embodiments, administration is oral.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrastemal injection, and infusion.

Actual dosage levels of the active ingredients in the pharmaceutical compositions provided herein may be varied so as to obtain an amount of the active ingredient which is effective to achieve the desired therapeutic response for a particular patient, composition, and mode of administration, without being toxic to the patient.

The concentration of a compound provided herein in a pharmaceutically acceptable mixture will vary depending on several factors, including the dosage of the compound to be administered, the pharmacokinetic characteristics of the compound(s) employed, and the route of administration. In some embodiments, the compositions provided herein can be provided in an aqueous solution containing about 0.1-10% w/v of a compound disclosed herein, among other substances, for parenteral administration. Typical dose ranges can include from about 0.01 to about 500 mg/kg of body weight per day, given in 1-4 divided doses. Each divided dose may contain the same or different compounds. The dosage will be a therapeutically effective amount depending on several factors including the overall health of a patient, and the formulation and route of administration of the selected compound(s).

Although the dosage will vary depending on the symptoms, age and body weight of the patient, the nature and severity of the disorder to be treated or prevented, the route of administration and the form of the drug, in general, a daily dosage of from 0.01 to 2000 mg of the compound is recommended for an adult human patient, and this may be administered in a single dose or in divided doses. The amount of active ingredient which can be combined with a carrier material to produce a single dosage form will generally be that amount of the compound which produces a therapeutic effect.

The precise time of administration and/or amount of the composition that will yield the most effective results in terms of efficacy of treatment in a given patient will depend upon the activity, pharmacokinetics, and bioavailability of a particular compound, physiological condition of the patient (including age, sex, disease type and stage, general physical condition, responsiveness to a given dosage, and type of medication), route of administration, etc. However, the above guidelines can be used as the basis for fine-tuning the treatment, e.g., determining the optimum time and/or amount of administration, which will require no more than routine experimentation consisting of monitoring the patient and adjusting the dosage and/or timing.

Also provided herein is a conjoint therapy wherein one or more other therapeutic agents are administered with a compound or a pharmaceutical composition comprising a compound provided herein. Such conjoint treatment may be achieved by way of the simultaneous, sequential, or separate dosing of the individual components of the treatment.

### Definitions

For the terms “for example” and “such as,” and grammatical equivalences thereof, the phrase “and without limitation” is understood to follow unless explicitly stated otherwise. As used herein, the term “about” is meant to account for variations due to experimental error. All measurements reported herein are understood to be modified by the term “about”, whether or not the term is explicitly used, unless explicitly stated otherwise. As used herein, the singular forms “a,” “an,” and “the” include plural referents unless the context clearly dictates otherwise.

A “subject,” as used herein, includes both humans and other animals, particularly mammals. Thus, the methods are applicable to both human therapy and veterinary applications. In some embodiments, the patient is a mammal, for example, a primate. In some embodiments, the patient is a human.

A “therapeutically effective” amount of a compound provided herein for a method involving treatment for a virus is typically one which is sufficient to prevent, eliminate, ameliorate or reduce the symptoms of a viral infection, including, but not limited to influenza, coronaviruses, respiratory syncytial virus (RSV), parainfluenza virus, human cytomegalovirus (HCMV) and adenovirus infection. It will be appreciated that different concentrations may be employed for prophylaxis than for treatment of an active disease.

A “virus production inhibiting” amount of a compound provided herein is typically one which is sufficient to achieve a measurable reduction in the amount of virus produced by the cells contacted with the compound. In some embodiments, a “virus production inhibiting” amount is an amount which inhibits a least 30% of the virus production in untreated cells. In some embodiments, a “virus production inhibiting” amount is an amount which inhibits a least 50% of the virus production in untreated cells. In some embodiments, a “virus production inhibiting” amount is an amount which inhibits a least 70% of the virus production in untreated cells. In some embodiments, a “virus production inhibiting” amount is an amount which inhibits a least 90% of the virus production in untreated cells.

A “therapeutically effective” amount of a compound provided herein for a method involving treatment for a tumor or a cancer is typically an amount effective to cause a reduction in the number of cancer cells in a patient or regression of a tumor is a patient relative to the size of the group of cancer cells or tumor prior to administration of the compound.

The terms “treatment” and “prevention” are art-recognized and include administration of one or more of the compounds or pharmaceutical compositions provided herein. If it is administered prior to clinical manifestation of the unwanted condition (e.g., disease or other unwanted state of the subject) then the treatment is preventative, (i.e., it protects the subject against developing the unwanted condition). As used in this context, the term “prevent” means to slow or prevent the onset of at least one symptom of a disorder as provided herein. For example, such prevention may be prompted by a likelihood of exposure to an infective agent (e.g., a virus) or when a subject exhibits other symptoms that indicate onset of a disorder (e.g., a metabolic disorder or cardiovascular disorder) may be likely. Alternatively, if it is administered after manifestation of the unwanted condition, the treatment is therapeutic, (i.e., it is intended to diminish, ameliorate, or stabilize the existing unwanted condition or side effects thereof). As used in this context, to “treat” means to ameliorate at least one symptom of a disorder as provided herein.

The term, “compound,” as used herein is meant to include all stereoisomers, geometric isomers, and tautomers of the structures depicted. Compounds herein identified by name or structure as one particular tautomeric form are intended to include other tautomeric forms unless otherwise specified.

In some embodiments, a compound provided herein, or salt thereof, is substantially isolated. By “substantially isolated” it is meant that the compound is at least partially or substantially separated from the environment in which it was formed or detected. Partial separation can include, for example, a composition enriched in the compound provided herein. Substantial separation can include compositions containing at least about 50%, at least about 60%, at least about 70%, at least about 80%, at least about 90%, at least about 95%, at least about 97%, or at least about 99% by weight of the compound provided herein, or salt thereof. Methods for isolating compounds and their salts are routine in the art.

The phrase “pharmaceutically acceptable” is used herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “pharmaceutically acceptable salt” refers to the relatively non-toxic, inorganic and organic acid addition salts of a compound provided herein. These salts can be prepared in situ during the final isolation and purification of a compound provided herein, or by separately reacting the compound in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, malonate, fumarate, succinate, tartrate, naphthylate, mesylate, glucoheptonate, lactobionate, laurylsulphonate salts, and amino acid salts, and the like. (See, for example, Berge et al. (1977) “Pharmaceutical Salts”, J. Pharm. Sci. 66: 1-19.)

In some embodiments, a compound provided herein may contain one or more acidic functional groups and, thus, is capable of forming pharmaceutically acceptable salts with pharmaceutically acceptable bases. The term “pharmaceutically acceptable salts” in these instances refers to the relatively non-toxic inorganic and organic base addition salts of a compound provided herein. These salts can likewise be prepared in situ during the final isolation and purification of the compound, or by separately reacting the purified compound in its free acid form with a suitable base, such as the hydroxide, carbonate, or bicarbonate of a pharmaceutically acceptable metal cation, with ammonia, or with a pharmaceutically acceptable organic primary, secondary, or tertiary amine. Representative alkali or alkaline earth salts include the lithium, sodium, potassium, calcium, magnesium, and aluminum salts, and the like. Representative organic amines useful for the formation of base addition salts include ethylamine, diethylamine, ethylenediamine, ethanolamine, diethanolamine, piperazine, and the like (see, for example, Berge et al., supra).

The term “solvate” means a compound that further includes a stoichiometric or non-stoichiometric amount of solvent bound by non-covalent intermolecular forces. Where the solvent is water, the solvate is a hydrate. The term “pharmaceutically acceptable solvate” refers to the relatively non-toxic solvates of a compound provided herein, using a solvent which is, within the sound scope of medical judgement, suitable for use in contact with the tissues of human beings and animals without excessive toxicity, irritation, allergic response or other problem or complication, commensurate with a reasonable benefit/risk ratio.

The term “alkyl” as employed herein refers to straight and branched chain aliphatic groups having from 1 to 12 carbon atoms, preferably 1-8 carbon atoms, and more preferably 1-6 carbon atoms, which is optionally substituted with one, two or three substituents. Preferred alkyl groups include, without limitation, methyl, ethyl, propyl, isopropyl, butyl, isobutyl, secbutyl, tertbutyl, pentyl, and hexyl. A “C₀” alkyl (as in “C₀-C₃-alkyl”) is a covalent bond (like “C₀” hydrocarbyl). The term “lower alkyl” refers to straight and branched chain aliphatic groups having from 1 to 6 carbon atoms. Unless otherwise specified, the term “alkyl” includes alkenyl, alkynyl and cyclic alkyl groups.

The term “alkenyl” as used herein means an unsaturated straight or branched chain aliphatic group with one or more carbon-carbon double bonds, having from 2 to 12 carbon atoms, preferably 2-8 carbon atoms, and more preferably 2-6 carbon atoms, which is optionally substituted with one, two or three substituents. Preferred alkenyl groups include, without limitation, ethenyl, propenyl, butenyl, pentenyl, and hexenyl.

The term “alkynyl” as used herein means an unsaturated straight or branched chain aliphatic group with one or more carbon-carbon triple bonds, having from 2 to 12 carbon atoms, preferably 2-8 carbon atoms, and more preferably 2-6 carbon atoms, which is optionally substituted with one, two or three substituents. Preferred alkynyl groups include, without limitation, ethynyl, propynyl, butynyl, pentynyl, and hexynyl.

The term “heteroalkyl” refers to an alkyl group, as defined herein above, wherein one or more carbon atoms in the chain are replaced by a heteroatom selected from the group consisting of O, S, and N.

An “aryl” group is a C₆-C₁₄ aromatic moiety comprising one to three aromatic rings, which is optionally substituted. Preferably, the aryl group is a C₆-C₁₀ aryl group. Preferred aryl groups include, without limitation, phenyl, naphthyl, anthracenyl, and fluorenyl.

A “heterocyclyl” or “heterocyclic” group is a ring structure having from about 3 to about 8 atoms, wherein one or more atoms are selected from the group consisting of N, O, and S. The heterocyclic group is optionally substituted on carbon at one or more positions. The heterocyclic group is also independently optionally substituted on nitrogen with alkyl, aryl, aralkyl, alkylcarbonyl, alkylsulfonyl, arylcarbonyl, arylsulfonyl, alkoxycarbonyl, aralkoxycarbonyl, or on sulfur with oxo or lower alkyl. Preferred heterocyclic groups include, without limitation, epoxy, aziridinyl, tetrahydrofuranyl, pyrrolidinyl, piperidinyl, piperazinyl, thiazolidinyl, oxazolidinyl, oxazolidinonyl, and morpholino. In certain preferred embodiments, the heterocyclic group is fused to an aryl, heteroaryl, or cycloalkyl group. Examples of such fused heterocyles include, without limitation, tetrahydroquinoline and dihydrobenzofuran. Specifically excluded from the scope of this term are compounds having adjacent annular O and/or S atoms.

As used herein, the term “heteroaryl” refers to groups having 5 to 14 ring atoms, preferably 5, 6, 9, or 10 ring atoms; having 6, 10, or 14 7C electrons shared in a cyclic array; and having, in addition to carbon atoms, from one to three heteroatoms per ring selected from the group consisting of N, O, and S. A “heteroaralkyl” or “heteroarylalkyl” group comprises a heteroaryl group covalently linked to an alkyl group, either of which is independently optionally substituted or unsubstituted. Preferred heteroaralkyl groups comprise a C1-C6 alkyl group and a heteroaryl group having 5, 6, 9, or 10 ring atoms. Specifically excluded from the scope of this term are compounds having adjacent annular O and/or S atoms. Examples of preferred heteroaralkyl groups include pyridylmethyl, pyridylethyl, pyrrolylmethyl, pyrrolylethyl, imidazolylmethyl, imidazolylethyl, thiazolylmethyl, and thiazolylethyl. Specifically excluded from the scope of this term are compounds having adjacent annular O and/or S atoms.

Embodiments of heterocyclyls and heteroaryls include, but are not limited to, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, 4aH-carb azolyl, carbolinyl, chromanyl, chromenyl, cinnolinyl, decahydroquinolinyl, 2H,6H-1,5,2-dithiazinyl, dihydrofuro[2,3b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl.

As employed herein, when a moiety (e.g., cycloalkyl, hydrocarbyl, aryl, heteroaryl, heterocyclic, urea, etc.) is described as “optionally substituted” it is meant that the group optionally has from one to four, preferably from one to three, more preferably one or two, non-hydrogen substituents. Suitable substituents include, without limitation, halo, hydroxy, oxo (e.g., an annular —CH— substituted with oxo is —C(O)—), nitro, halohydrocarbyl, hydrocarbyl, aryl, aralkyl, alkoxy, aryloxy, amino, acylamino, alkylcarbamoyl, arylcarbamoyl, aminoalkyl, acyl, carboxy, hydroxyalkyl, alkanesulfonyl, arenesulfonyl, alkanesulfonamido, arenesulfonamido, aralkylsulfonamido, alkylcarbonyl, acyloxy, cyano, and ureido groups.

As employed herein, a “heteroatom” is a nitrogen, sulfur or oxygen atom that has replaced a carbon atom in an alkyl, alkenyl, alkynyl, alkoxy, cycloalkyl, cycloalkoxy, aryl, or other hydrocarbon molecular structure.

The term “halogen” or “halo” as employed herein refers to chlorine, bromine, fluorine, or iodine. As herein employed, the term “acyl” refers to an alkylcarbonyl or arylcarbonyl substituent. The term “acylamino” refers to an amide group attached at the nitrogen atom (i.e., R—CO—NH—). The term “carbamoyl” refers to an amide group attached at the carbonyl carbon atom (i.e., NH₂—CO—). The nitrogen atom of an acylamino or carbamoyl substituent is optionally additionally substituted. The term “sulfonamido” refers to a sulfonamide substituent attached by either the sulfur or the nitrogen atom. The term “amino” is meant to include NH₂, alkylamino, arylamino, and cyclic amino groups. The term “ureido” as employed herein refers to a substituted or unsubstituted urea moiety.

A moiety that is substituted is one in which one or more hydrogens have been independently replaced with another chemical substituent. As a non-limiting example, substituted phenyls include 2-flurophenyl, 3,4-dichlorophenyl, 3-chloro-4-fluoro-phenyl, 2-fluor-3-propylphenyl. As another non-limiting example, substituted n-octyls include 2,4 dimethyl-5-ethy-octyl and 3-cyclopentyl-octyl. Included within this definition are methylenes (—CH₂—) substituted with oxygen to form carbonyl-CO—).

An “unsubstituted” moiety as defined above (e.g., unsubstituted cycloalkyl, unsubstituted heteroaryl, etc.) means that moiety as defined above that does not have any of the optional substituents for which the definition of the moiety (above) otherwise provides. Thus, for example, while an “aryl” includes phenyl and phenyl substituted with a halo, “unsubstituted aryl” does not include phenyl substituted with a halo.

### Synthesis of Compounds of the Invention

The compounds in the present invention (compounds of Formula I) can be prepared using the general reaction scheme set out in the schemes below. The following abbreviations are used: NMP, N-methyl-2-pyrrolidone; RT, room temperature; DCM, dichloromethane; DMF, N,N-Dimethylformamide; THF, tetrahydrofuran; DCE, 1,2-dichloroethane; TES or TES-H, triethylsilane; TES, triethoxysilane; TFA, trifluoroacetic acid; EtOAc or EA, ethyl acetate; M, molar; TBAF, tetrabutylammonium fluoride; t-BuOH, t-butanol; MeI, methyl iodide; DMSO, dimethylsulfoxide; MeCN, acetonitrile; XPhos, 2-dicyclohexylphosphino-2′,4′,6′-triisopropylbiphenyl; MeOH, methanol; h or hrs, hours; aq., aqueous; DME, 1,2-dimethoxyethane; sat., saturated; atm, atmosphere; Ac₂O, acetic anhydride; conc., concentrated; eq., equivalents; DIEA, N,N-diisopropylethylamine; HATU, N-[(Dimethylamino)-1H-1,2,3-triazolo-[4,5-b]pyridin-1-ylmethylene]-N-methyl-methanaminium hexafluorophosphate N-oxide; DMA, N,N-Dimethylacetamide; Pd₂(dba)₃, tris(dibenzylideneacetone)dipalladium(0); S-Phos, dicyclohexyl(2′,6′-dimethoxy-[1,1′-biphenyl]-2-yl)phosphine; PE, peteroleum ether; AcOK, potassium acetate; Pd(dppf)Cl₂, [1,1′-Bis(diphenylphosphino)ferrocene]dichloropalladium(II); DMI, 1,3-dimethyl-2-imidazolidinone; Prep-TLC, preperative thin layer chromatography; t-BuONa, sodium t-butoxide; t-BuOK, potassium t-butoxide; HMDS, hexamethyldisilazane; (Pd(OAc)₂, palladium (II) acetate; EtOH, ethanol; DEA, diethylamine; AcOH, acetic acid; BOC₂O, di-tert-butyl dicarbonate; Et₃N, triethylamine; Prep-HPLC, preparative HPLC; TsOH, p-toluenesulfonic acid; TBAB, Tetra-n-butylammonium bromide.

The compounds in the present invention can be prepared using the general reaction scheme set out in the schemes below.

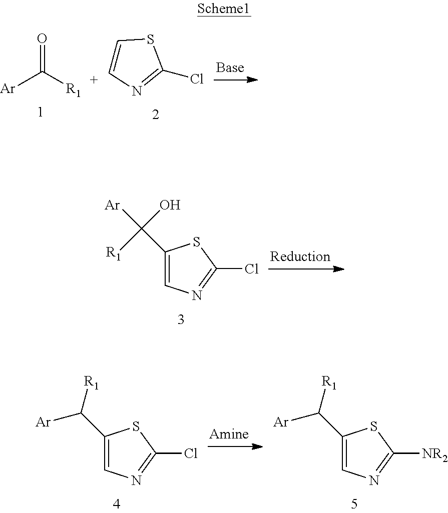

A base, e.g., n-BuLi or sec-BuLi can be reacted with 2-chloro-1,3-thiazole (2) and a suitable aromatic or heteroaromatic aldehyde or ketone of general formula 1 to afford compounds of general structure 3. Compounds of general structure 3 can be treated with a suitable reducing agent, e.g., a silane such as triethylsilane and an acid such as trifluoroacetic acid to provide compounds of general formula 4. Compounds of general formula 4 can be treated with a suitable amine, e.g., a substituted or unsubstituted 1,2,3,4-tetrahydroisoquinoline or a substituted or unsubstituted 5,6,7,8-tetrahydro-1,6-naphthyridine to afford compounds of general formula 5. It will be recognized that compounds of general formula 5 are identical to compounds of Formula I.

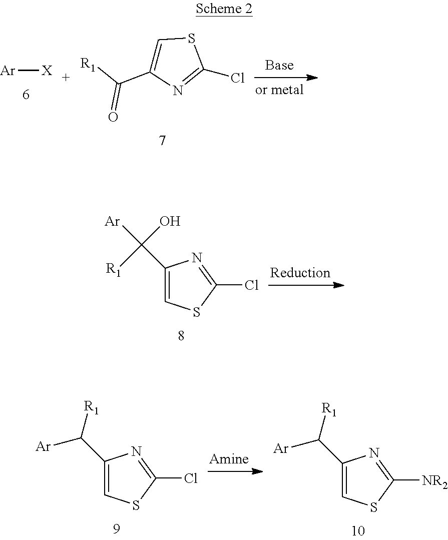

A base, e.g., n-BuLi or sec-BuLi or a metal, e.g., Mg or Li, can be reacted with a suitable halogenated aromatic or heteroaromatic of general formula 6, where X is Cl, Br or I, and compounds of general formula 7 to afford compounds of general formula 8. Compounds of general formula 8 can be treated with a suitable reducing agent, e.g., a silane such as triethylsilane and an acid such as trifluoroacetic acid to provide compounds of general formula 9. Compounds of general structure 9 can be treated with a suitable amine, e.g., a substituted or unsubstituted 1,2,3,4-tetrahydroisoquinoline or a substituted or unsubstituted 5,6,7,8-tetrahydro-1,6-naphthyridine to afford compounds of general formula 10. It will be recognized that compounds of general formula 10 are identical to compounds of Formula I.

Those skilled in the art will recognize there may be alternate synthetic paths to provide compounds of Formula I. The following Schemes describe examples of such alternate synthetic paths but are not to be considered limiting.

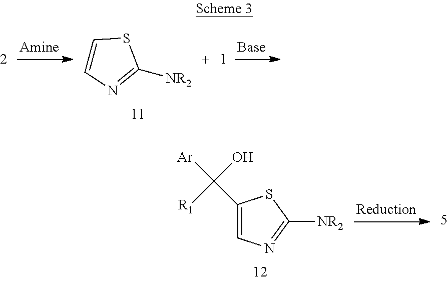

In some instances, a suitable amine, e.g., a substituted or unsubstituted 1,2,3,4-tetrahydroisoquinoline or a substituted or unsubstituted 5,6,7,8-tetrahydro-1,6-naphthyridine can be reacted with 2-chloro-1,3-thiazole (2) to afford compounds of general formula 11. Compounds of general formula 11 can be reacted with base, e.g., n-BuLi or sec-BuLi and compounds of general formula 1 to afford compounds of general formula 12. Compounds of general formula 12 can be treated with a suitable reducing agent, e.g., a silane such as triethylsilane and an acid such as trifluoroacetic acid to provide compounds of general formula 5.

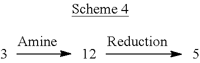

In some instances, compounds of general formula 3 can be treated with a suitable amine, e.g., a substituted or unsubstituted 1,2,3,4-tetrahydroisoquinoline or a substituted or unsubstituted 5,6,7,8-tetrahydro-1,6-naphthyridine to afford compounds of general formula 12. Compounds of general formula 12 can be treated as described above to provide compound of general formula 5.

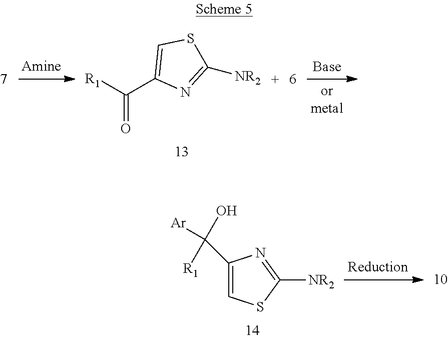

In some instances, a suitable amine, e.g., a substituted or unsubstituted 1,2,3,4-tetrahydroisoquinoline or a substituted or unsubstituted 5,6,7,8-tetrahydro-1,6-naphthyridine can be reacted with compounds of general formula 7 to afford compounds of general formula 13. A base, e.g., n-BuLi or sec-BuLi or a metal, e.g., Mg or Li, can be reacted with a suitable halogenated aromatic or heteroaromatic compounds of general formula 6, where X is Cl, Br or I, and compounds of general formula 13 to afford compounds of general formula 14. Compounds of general formula 12 can be treated as described above to provide compound of general formula 10.

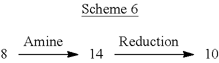

In some instances, compounds of general formula 8 can be reacted with a suitable amine, e.g., a substituted or unsubstituted 1,2,3,4-tetrahydroisoquinoline or a substituted or unsubstituted 5,6,7,8-tetrahydro-1,6-naphthyridine to afford compounds of general formula 14. Compounds of general formula 14 can be treated as described above to provide compound of general formula 10.

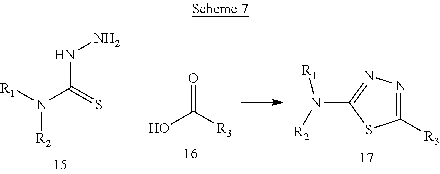

A thiosemicarbazide of general formula 15, where R1 and R2 may comprise a substituted or unsubstituted 1,2,3,4-tetrahydroisoquinoline or a substituted or unsubstituted 5,6,7,8-tetrahydro-1,6-naphthyridine, and a carboxylic acid of general structure 16 can be treated with a dehydrating agent, e.g., POCl₃, followed by optional heating to form compounds of general formula 17. It will be recognized that compounds of general formula 17 are identical to compounds of Formula I.

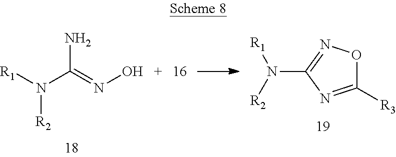

A N-hydroxyguanidine of general formula 18, where R1 and R2 may comprise a substituted or unsubstituted 1,2,3,4-tetrahydroisoquinoline or a substituted or unsubstituted 5,6,7,8-tetrahydro-1,6-naphthyridine, and a carboxylic acid of general structure 16 can be treated with a dehydrating agent, e.g., 1,1′-carbonyldiimidazole, followed by optional heating in an appropriate solvent, e.g., DMF, toluene and the like, to form compounds of general formula 19. It will be recognized that compounds of general formula 19 are identical to compounds of Formula I.

Methods to perform the above described reactions and processes would be apparent to those of ordinary skill in the art based on the present disclosure, or can be deduced in analogy from the examples. Starting materials are commercially available or can be made by methods analogous to those described in the Examples below.

### EXAMPLES

### Preparation of Intermediates

### Preparation of Intermediate 1

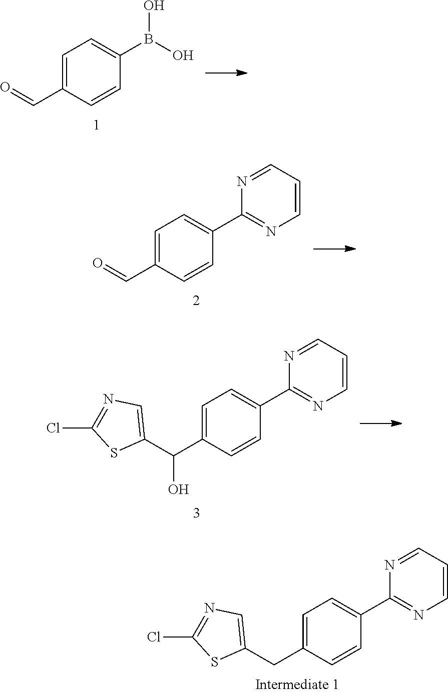 [SMILES: O=Cc1ccc(B(O)O)cc1 ; O=Cc1ccc(-c2ncccn2)cc1 ; OC(c1ccc(-c2ncccn2)cc1)c1cnc(Cl)s1 ; Clc1ncc(Cc2ccc(-c3ncccn3)cc2)s1] [0220] 1. A mixture of 1 (Sigma-Aldrich, 4 g, 26.7 mmol), 2-bromopyrimidine (3.51 g, 22.1 mmol), NaHCO₃ (6.73 g, 80.1 mmol), Pd(PPh₃)₄ (766 mg, 0.663 mmol) in DME/H₂O (100 mL/50 mL) was stirred at 90° C. for 18 h under N₂. The mixture was filtered through Celite and the filter cake washed with EA and the filtrate was concentrated. The resulting mixture was diluted with EA and the combined extracts were washed with brine, water, and dried with Na₂SO₄. The mixture was filtered and the filtrate concentrated to give a residue which was purified by silica gel chromatography to afford 2 (3.84 g, 78.1% yield) as a white solid. MS (ESI): mass calcd. for C₁₁H₈N₂O 184.20, m/z found 185.0 [M+H]⁺.
2. To a solution of 2-chlorothiazole (2.69 g, 22.7 mmol) in dry THF (80 mL) at −78° C. under N₂ n-BuLi (2.4 M, 9.9 mL, 23.75 mmol) was added dropwise. After 1 h a solution of 2 (3.8 g, 20.6 mmol, 106 mL THF) was added dropwise to the mixture. The reaction was warmed to RT and stirred for 18 h. The resulting mixture was diluted with aq. NH₄Cl and extracted with EtOAc and the combined extracts were dried over Na₂SO₄, filtered and the filtrate concentrated to give a residue. The residue was purified by silica gel chromatography to afford 3 (4.65 g, 74.2% yield) as a white solid. MS (ESI): mass calcd. for C₁₄H₁₀ClN₃OS 303.76, m/z found 303.8 [M+H]⁺.
3. To a solution of 3 (3 g, 9.87 mmol) and TFA (11.3 g, 98.7 mmol) in dry DCE at 0° C. TES (3.42 g, 29.6 mmol) was added dropwise and the reaction was stirred for 8 h at 60° C. The mixture was cooled and concentrated. The resulting residue was treated with saturated NaHCO₃, extracted with EA and the combined extracts were washed with brine, water and dried with Na₂SO₄. The mixture was filtered and the filtrate concentrated to give a residue which was purified by flash chromatography to afford Intermediate 1 (1.2 g, 42.2% yield) as a white solid. MS (ESI): mass calcd. for C₁₄H₁₀ClN₃S 287.77, m/z found 287.8 [M+H]⁺.

### Intermediate 2

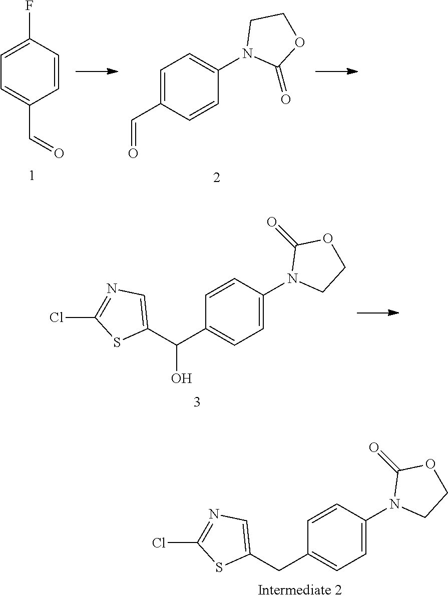 [SMILES: O=Cc1ccc(F)cc1 ; O=Cc1ccc(N2CCOC2=O)cc1 ; O=C1OCCN1c1ccc(C(O)c2cnc(Cl)s2)cc1 ; O=C1OCCN1c1ccc(Cc2cnc(Cl)s2)cc1] [0224] 1. To a solution of 1 (4.46 g, 36 mmol) in DMF (100 mL) was added oxazolidin-2-one (2.61 g, 30 mmol) and Cs₂CO₃ (11.74 g, 36 mmol). The resulting mixture was stirred at 120° C. overnight. The mixture was cooled to RT, filtered, the filtrate poured into water and the mixture extracted with EA. The combined extracts were washed with brine, dried with Na₂SO₄, concentrated and the resulting residue purified by chromatography on silica gel to give 2 (4.17 g, 70% yield) as a white solid. MS (ESI): mass calcd. for C₁₀H₉NO₃ 191.19, m/z found 192.0 [M+H]⁺.
2. To a solution of 2-chlorothiazole (0.86 g, 7.2 mmol) in THF (25 mL) at −78° C. was added n-BuLi (3 mL, 7.2 mmol) dropwise. After 1 h, a solution of 2 (1.06 g, 5.5 mmol) in THF (15 mL) was added dropwise. The reaction mixture was warmed to RT stirred 2 h, diluted with sat. aq. NH₄Cl and extracted with EtOAc. The combined extracts were washed with brine, dried over Na₂SO₄, filtered, concentrated to give 3 (1.8 g) as a yellow oil which was used without purification in the next step. MS (ESI): mass calcd. for C₁₄H₁₂ClN₃O₂S 310.75, m/z found 311.21[M+H]⁺.
3. A mixture of 3 (0.1 g, 0.32 mmol) and TES (0.5 mL) in TFA (1 mL) was stirred at RT for 2 h. The mixture was concentrated and the residue was purified by chromatography on silica gel to give Intermediate 2 (78 mg, 82% yield) as a white solid. MS (ESI): mass calcd. for C₁₃H₁₁—ClN₂O₂S 294.75, m/z found 295.19[M+H]⁺.

### Intermediate 3

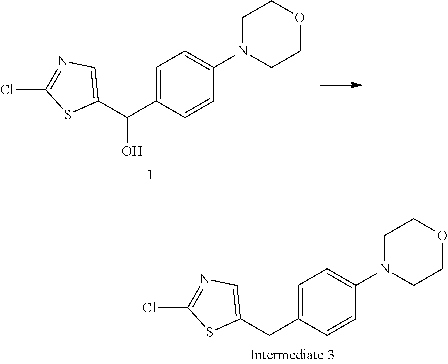 [SMILES: OC(c1ccc(N2CCOCC2)cc1)c1cnc(Cl)s1 ; Clc1ncc(Cc2ccc(N3CCOCC3)cc2)s1] [0228] 1. A solution of 2-chlorothiazole (1.76 g, 14.7 mmol) in dry THF (50 mL) was cooled to −78° C. and n-BuLi (2.4 M in Hex, 6.4 mL, 15.3 mmol) was added dropwise. After 1 h, a solution of 4-morpholinobenzaldehyde (Sigma-Aldrich, 2.50 g, 13.3 mmol) in THF (70 mL) was added dropwise to the mixture at −78° C. The reaction was warmed to room temperature and stirred for 18 h, diluted with aq. NH₄Cl (100 mL) and extracted with EtOAc. The combined extracts were dried over Na₂SO₄, filtered and the filtrate concentrated to give 1 (5.00 g) as a yellow oil which was used in the next step without purification.
2. TES (7.76 g, 68.00 mmol) was added dropwise to a solution of 1 (5.0 g, 17.0 mmol) in dry TFA (50 mL) at RT and the mixture was stirred for 2 h at 80° C. The mixture was cooled, concentrated and the residue treated with saturated aq. NaHCO₃. The resulting mixture was extracted with EA and the combined extracts washed with brine, water, and then dried with Na₂SO₄. The mixture was concentrated and the residue purified by flash chromatography to afford Intermediate 3 (3.0 g) as a white solid.

### Intermediate 4

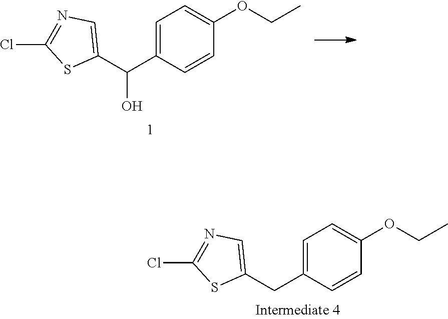 [SMILES: CCOc1ccc(C(O)c2cnc(Cl)s2)cc1 ; CCOc1ccc(Cc2cnc(Cl)s2)cc1] [0231] 1. Following the procedure described for Intermediate 1 Step 2, 1.76 g of 2-chlorothiazole and 2 g of 4-ethoxybenzaldehyde were converted to 1 (2.8 g, 78% yield) obtained as a white solid. MS (ESI): mass calcd. for C₁₂H₁₂ClNO₂S 269.74, m/z found 269.9 [M+H]⁺.
2. Following the procedure described for Intermediate 3 Step 2 except the reaction was heated at 70° C. for 2 h, 1 g of 1 was treated with 1.69 g of TES to give Intermediate 4 (0.8 g 85% yield) which was obtained as a white solid. MS (ESI): mass calcd. for C₁₂H₁₂ClNOS 253.74, m/z found 253.9 [M+H]⁺.

### Intermediate 5

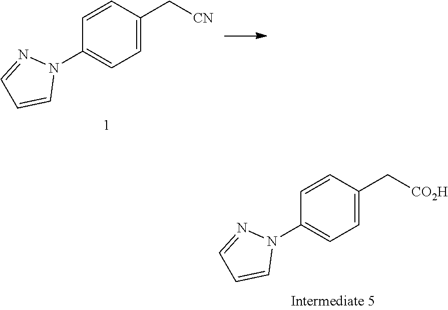 [SMILES: N#CCc1ccc(-n2cccn2)cc1 ; O=C(O)Cc1ccc(-n2cccn2)cc1] [0234] 1. A mixture of 1-[4-(chloromethyl)phenyl]-1H-pyrazole (Sigma-Aldrich, 2.00 g, 10.38 mmol), TMSCN (2.05 mg, 20.76 mmol) and K₂CO₃ (28.6 g, 20.76 mmol) in CH₃CN (10 mL) was stirred at 110° C. under microwave irradiation for 1 h. The mixture was diluted with water, extracted with EtOAc and the combined organic extracts washed with water, brine and dried over Na₂SO₄. The mixture was filtered and the filtrate concentrated to give a residue which was purified by silica gel column chromatography to afford 1 (1.2 g) as a white solid.
2. A mixture of 1 (1.2 g, 6.55 mmol) and conc. HCl (8 mL) was stirred at 110° C. for 1 h. The mixture was concentrated, the resulting solid material collected by filtration and washed with water to afford Intermediate 5 (800 mg) as a yellow solid.

### Intermediate 6

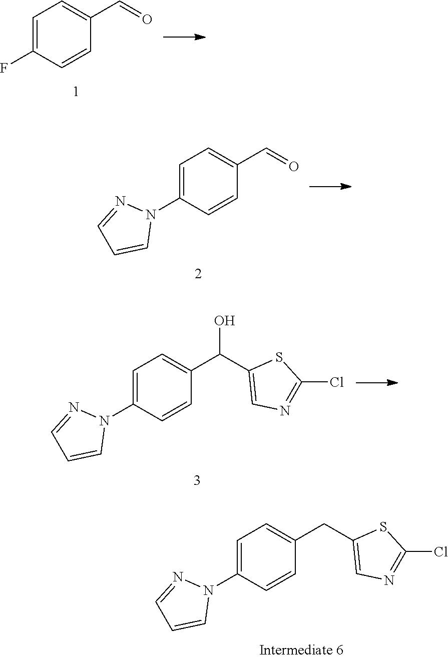 [SMILES: O=Cc1ccc(F)cc1 ; O=Cc1ccc(-n2cccn2)cc1 ; OC(c1ccc(-n2cccn2)cc1)c1cnc(Cl)s1 ; Clc1ncc(Cc2ccc(-n3cccn3)cc2)s1] [0237] 1. To a solution of 1 (10 g, 80.6 mmol) in dry DMF (100 mL) were added pyrazole (5.5 g, 80.6 mmol) and K₂CO₃ (12.2 g, 88.7 mmol). The resulting mixture was stirred at 100° C. overnight. After cooling to RT, the mixture was diluted with water and extracted with EA. The organic extracts were washed with water, brine, dried over anhydrous Na₂SO₄, filtered and the filtrate concentrated to give an oil. The material was purified by recrystallization to afford 2 (4 g, 29%). ¹HNMR (CDCl₃, 300 MHz) δ: 6.5-6.6 (s, 1 H), 7.7-7.8 (s, 1 H), 7.9-8.0 (d, 2 H), 8.0-8.1 (d, 2 H), 8.1-8.2 (s, 1 H), 10.0-10.1 (s, 1 H).
2. To a solution of 2-chlorothiazole (1.45 g, 12.1 mmol) in dry THF (10 mL) at −78° C. under N₂ was added n-BuLi (5 mL, 12.1 mmol) dropwise. After 1 h a solution of 2 (1.6 g, 9.3 mmol) was added dropwise at −78° C. The resulting solution was warmed to RT. The mixture was diluted with sat. NH₄Cl and extracted with EA. The combined extracts were concentrated to give a residue which was purified by silica gel chromatography to afford 3 (1.2 g, 50%). ¹HNMR (CDCl₃, 300 MHz) δ: 6.1-6.2 (s, 1 H), 6.5-6.6 (s, 1 H), 7.2-7.3 (s, 1 H), 7.4-7.5 (d, 2 H), 7.6-7.7 (d, 2 H), 7.7-7.8 (s, 1 H), 7.9-8.0 (s, 1 H).
3. To a solution of 3 (1.2 g, 4.1 mmol) in DCE (20 mL) was added TES (1.4 g, 12.8 mmol), the mixture cooled to 0° C. and TFA (4.7 g, 41 mmol) was added dropwise. The resulting solution was stirred at 60° C. for 4 h. The mixture was concentrated and the residue purified by silica gel chromatography to afford Intermediate 6 (1 g, 91%). ¹HNMR (CDCl₃, 300 MHz) δ: 4.1-4.2 (s, 2 H), 6.4-6.5 (s, 1 H), 7.2-7.4 (m, 3 H), 7.6-7.8 (m, 3 H), 7.9-8.0 (s, 1 H).

### EXAMPLES

### Example 001

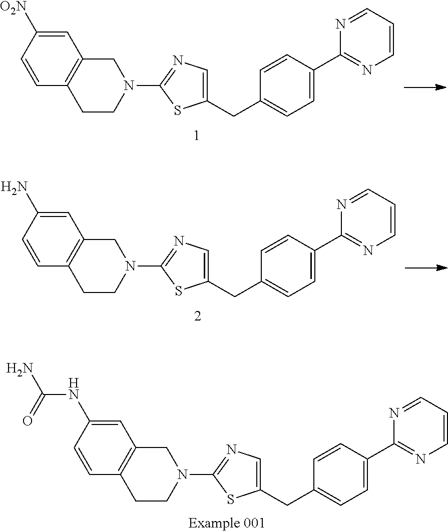 [SMILES: O=[N+]([O-])c1ccc2c(c1)CN(c1ncc(Cc3ccc(-c4ncccn4)cc3)s1)CC2 ; Nc1ccc2c(c1)CN(c1ncc(Cc3ccc(-c4ncccn4)cc3)s1)CC2 ; NC(=O)Nc1ccc2c(c1)CN(c1ncc(Cc3ccc(-c4ncccn4)cc3)s1)CC2] [0241] 1. A mixture of Intermediate 1 (300 mg, 1.04 mmol), 7-nitro-1,2,3,4-tetrahydroisoquinoline (186 mg, 1.04 mmol), Cs₂CO₃ (1.02 g, 3.13 mmol), Pd₂(dba)₃ (477 mg, 0.52 mmol) and S-Phos (213 mg, 0.52 mmol) in 1,4-dioxane (15 mL) was evacuated and refilled with N₂ three times. The resulting mixture was stirred at 100° C. for 15 hours, cooled to room temperature, diluted with DCM/MeOH (10:1, 20 mL), filtered and the filtrate was concentrated. The resulting residue was purified by chromatography on silica gel to give 1 (350 mg, 78% yield) as a white solid. MS (ESI): mass calcd. for C₂₃H₁₉N₅O₂S 429.50, m/z found 429.8 [M+H]⁺.
2. To a mixture 1 (350 mg, 0.82 mmol) in MeOH (15 mL) and DCM (5 mL) was added NH₄Cl (439 mg, 8.2 mmol) and Zn (267 mg, 4.1 mmol). The resulting mixture was stirred at room temperature for 16 hours. The mixture was concentrated and the residue was diluted with aqueous NH₃ (15 mL) and extracted with ethyl acetate. The combined extracts were washed with brine, dried over Na₂SO₄, filtered, concentrated and the residue purified by chromatography on silica gel to give 2 (200 mg, 61.5% yield) as a pale yellow solid. MS (ESI): mass calcd. for C₂₃H₂₁N₅S 399.52, m/z found 399.8 [M+H]⁺.
3. A mixture of 2 (100 mg, 0.25 mmol), KCNO (40 mg, 0.5 mmol) in HOAc (1 mL) and H₂O (1 mL) was stirred at 25° C. for 2 hours. The mixture was diluted with EA, washed with water, brine and dried over Na₂SO₄, filtered and the filtrate concentrated. The residue was purified by Prep-HPLC to give Example 001 (5.57 mg, 5% yield) as a white solid. MS (ESI): mass calcd. for C₂₄H₂₂N₆OS 442.54, m/z found 442.8 [M+H]⁺. ¹H NMR (400 MHz, DMSO) δ ppm 8.90 (d, J=4.8 Hz, 2H), 8.49 (s, 1H), 8.34 (d, J=7.9 Hz, 2H), 7.45-7.40 (m, 3H), 7.26 (s, 1H), 7.18 (d, J=8.4 Hz, 1H), 7.04-7.01 (m, 2H), 5.83 (s, 2H), 4.48 (s, 2H), 4.09 (s, 2H), 3.63 (t, J=5.8 Hz, 2H), 2.80 (t, J=5.8 Hz, 2H).

### Example 002

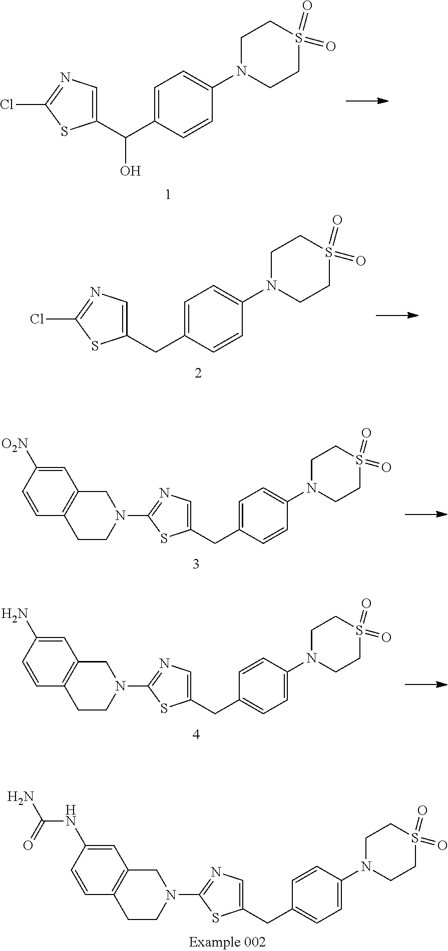 [SMILES: O=S1(=O)CCN(c2ccc(C(O)c3cnc(Cl)s3)cc2)CC1 ; O=S1(=O)CCN(c2ccc(Cc3cnc(Cl)s3)cc2)CC1 ; O=[N+]([O-])c1ccc2c(c1)CN(c1ncc(Cc3ccc(N4CCS(=O)(=O)CC4)cc3)s1)CC2 ; Nc1ccc2c(c1)CN(c1ncc(Cc3ccc(N4CCS(=O)(=O)CC4)cc3)s1)CC2 ; NC(=O)Nc1ccc2c(c1)CN(c1ncc(Cc3ccc(N4CCS(=O)(=O)CC4)cc3)s1)CC2] [0245] 1. To a solution of 2-chlorothiazole (1.02 g, 8.52 mmol) in dry THF (50 mL) at −78° C. under N₂ was added, dropwise, n-BuLi (2.5 M in Hex, 3.9 mL, 9.8 mmol). After 1 hour, a solution of 4-(1,1-dioxide-4-thiomorpholine)benzaldehyde (TCI, 1.70 g, 7.10 mmol) in THF (10 mL) was added dropwise to the mixture at −78° C. The reaction was slowly warmed to room temperature, stirred for 18 h, diluted with aq. NH₄Cl and extracted with EA. The combined extracts were washed with brine, dried over Na₂SO₄, filtered and the filtrate concentrated to give a residue which was purified by silica gel chromatography to afford 1 (900 mg) as a white solid.
2. TES (390 mg, 3.36 mmol) was added dropwise to a solution of 1 (300 mg, 0.84 mmol) in dry TFA (5 mL) at room temperature and the mixture stirred for 2 h at 80° C. The solution was concentrated, the residue was treated with saturated aq. NaHCO₃ and extracted with EA. The combined organic phase was washed with brine, dried over Na₂SO₄ filtered and the filtrate concentrated. The resulting residue was purified by silica gel chromatography to afford 2 (240 mg) as a white solid.
3. A mixture of 2 (240 mg, 0.70 mmol), 7-nitro-1,2,3,4-tetrahydroisoquinoline (124 mg, 0.70 mmol), Pd₂(dba)₃ (64 mg, 0.07 mmol), S-Phos (28 mg, 0.07 mmol), Cs₂CO₃ (342 mg, 1.05 mmol) in dioxane (10 mL) was stirred at 100° C. for 16 hours. The mixture was cooled to room temperature, diluted with water and extracted with EA. The combined extracts were washed with water, brine, dried over Na₂SO₄ filtered and the filtrate concentrated. The residue was purified by silica gel column chromatography to afford 3 (260 mg) as white solid.
4. A mixture of 3 (260 mg, 0.54 mmol), Zn (70 mg, 1.08 mmol), NH₄Cl (57 mg, 1.08 mmol) in MeOH (10 mL) was stirred at 70° C. for 2 hours. The mixture was cooled, filtered and the filtrate concentrated. The resulting residue was diluted with EA, washed with water, brine and dried over Na₂SO₄. The mixture was filtered and the filtrate concentrated to afford 4 (200 mg) as yellow oil which was used directly in next step without further purification.
5. Following the procedure described for Example 001, Step 3 except 1.5 mL HOAc and 1 mL H₂O were used, 200 mg of 4 and 35 mg of KCNO were reacted to afford Example 002 (25 mg, 6% yield) as a white solid. ¹H NMR (500 MHz, DMSO-d6) δ 8.53 (s, 1H), 7.26 (s, 1H), 7.16 (s, 1H), 7.11 (d, J=8.1 Hz, 2H), 7.01 (d, J=8.2 Hz, 1H), 6.96 (d, J=7.1 Hz, 3H), 5.86 (s, 2H), 4.45 (s, 2H), 3.88 (s, 2H), 3.72 (s, 4H), 3.61 (t, J=5.7 Hz, 2H), 3.10 (s, 4H), 2.79 (t, J=5.5 Hz, 2H).

### Example 003

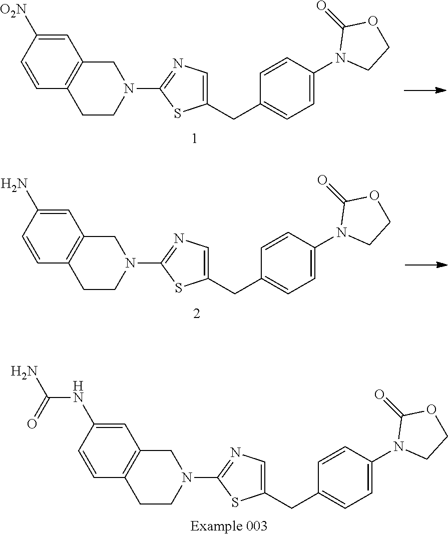 [SMILES: O=C1OCCN1c1ccc(Cc2cnc(N3CCc4ccc([N+](=O)[O-])cc4C3)s2)cc1 ; Nc1ccc2c(c1)CN(c1ncc(Cc3ccc(N4CCOC4=O)cc3)s1)CC2 ; NC(=O)Nc1ccc2c(c1)CN(c1ncc(Cc3ccc(N4CCOC4=O)cc3)s1)CC2] [0251] 1. A mixture of Intermediate 2 (900 mg, 3.0 mmol), 7-nitro-1,2,3,4-tetrahydroisoquinoline (1.60 g, 9.0 mmol), Pd(dba)₂ (870 mg, 1.5 mmol), S-Phos (630 mg, 1.5 mmol) and Cs₂CO₃ (2.940 g, 9.0 mmol) in dried 1,4-dioxane (50 mL) was stirred at 100° C. overnight. The mixture was cooled to RT, filtered and the filtrate concentrated to afford a residue which was purified by silica gel chromatography to afford 1 (1.50 g) as an oil. MS (ESI): mass calcd. for C₂₂H₂₀N₄O₄S 436.12. m/z found 436.9 [M+H]⁺.
2. A mixture of 1 (880 mg, 2.0 mmol), Zn (1.30 g, 20 mmol) and NH₄Cl (1.08 g, 20 mmol) in DCM (20 mL) and MeOH (20 mL) was stirred at RT overnight. The mixture was filtered, the filtrate concentrated to afford a residue which was purified by silica gel chromatography to give 2 (1.50 g) as a solid. MS (ESI): mass calcd. for C₂₂H₂₂N₄O₂S 406.15. m/z found 406.9 [M+H]⁺.
3. A mixture of 2 (243 mg, 0.6 mmol) and KOCN (99 mg, 1.2 mmol) in AcOH (4 mL) and H₂O (4 mL) was stirred at 0° C. for 1 hour, poured into water, neutralized with NaHCO₃ (aq). The resulting mixture was extracted with EA, the combined extracts dried over Na₂SO₄, filtered and the filtrate concentrated. The resulting residue was purified by Prep-HPLC to afford Example 003 as white solid (40 mg, 15%). MS (ESI): mass calcd. for C₂₃H₂₃N₅O₃S 449.15. m/z found 449.9 [M+H]⁺. ¹H NMR (400 MHz, DMSO-d6) δ ppm 8.51 (s, 1H), 7.52 (d, J=8.6 Hz, 2H), 7.39-7.26 (m, 3H), 7.18 (dd, J=12.6, 4.5 Hz, 2H), 7.06 (d, J=8.3 Hz, 1H), 5.85 (s, 2H), 4.54 (s, 2H), 4.50-4.37 (m, 2H), 4.04 (m, 4H), 3.65 (t, J=5.9 Hz, 2H), 2.85 (t, J=5.7 Hz, 2H).

### Example 004

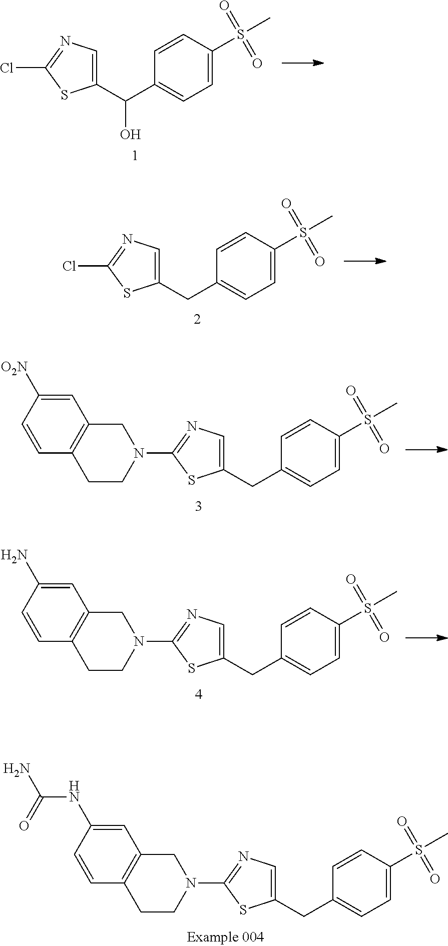 [SMILES: CS(=O)(=O)c1ccc(C(O)c2cnc(Cl)s2)cc1 ; CS(=O)(=O)c1ccc(Cc2cnc(Cl)s2)cc1 ; CS(=O)(=O)c1ccc(Cc2cnc(N3CCc4ccc([N+](=O)[O-])cc4C3)s2)cc1 ; CS(=O)(=O)c1ccc(Cc2cnc(N3CCc4ccc(N)cc4C3)s2)cc1 ; CS(=O)(=O)c1ccc(Cc2cnc(N3CCc4ccc(NC(N)=O)cc4C3)s2)cc1] [0255] 1. To a solution of 2-chlorothiazole (3.59 g, 29.9 mmol) in dry THF (94 mL) at −78° C. under N₂ was added dropwise n-BuLi (2.4 M in Hex, 13.0 mL, 31.2 mmol). After 1 h, a solution of 4-methanesulfonylbenzaldehyde (Enamine, 5 g, 27.2 mmol) in dried THF (200 mL) was added dropwise. The reaction was warmed to RT and stirred for 18 h. The mixture was diluted with saturated aq. NH₄Cl and the mixture extracted with EtOAc. The combined extracts were dried over Na₂SO₄, filtered and the filtrate concentrated to give a residue which was purified by silica gel chromatography to afford 1 (1.6 g, 19.4% yield) as a white solid. MS (ESI): mass calcd. for C₁₁H₁₀ClNO₃S₂ 303.78, m/z found 303.7 [M+H]⁺.
2. To a solution of 1 (1.6 g, 5.26 mmol) in TFA (12 mL) was added dropwise TES (3 g, 26.3 mmol) over 15 min. The reaction was stirred at 70° C. for 2 h, diluted with sat. NaHCO₃ and the mixture extracted with EtOAc. The combined extracts were washed with water, dried over Na₂SO₄, filtered and the filtrate concentrated to give a crude product which was purified by silica gel chromatography to afford 2 (1.3 g, 85.8% yield) as a white solid. MS (ESI): mass calcd. for C₁₁H₁₀ClNO₂S₂ 287.78, m/z found 287.7 [M+H]⁺.
3. A mixture of 2 (445 mg, 1.545 mmol), 7-nitro-1,2,3,4-tetrahydroisoquinoline (275 mg, 1.545 mmol), Cs₂CO₃ (1.511 g, 4.635 mmol), Pd(dba)₂ (444 mg, 0.772 mmol) and S-Phos (317 mg, 0.772 mmol) in dried dioxane (60 mL) was stirred at 100° C. for 5 h under N₂. The mixture was cooled to RT, diluted with DCM:MeOH (10:1, 20 mL), filtered and the filter cake washed with DCM:MeOH (10:1). The combined filtrate was concentrated to afford a residue which was purified by combi-Flash to give 3 (340 mg, 51.2%) as a yellow solid. MS (ESI): mass calcd. for C₂₀H₁₉N₃O₄S₂ 429.51 m/z found 430.0 [M+H]⁺.
4. Following the procedure described for Example 003, Step 2, except the crude product was purified by silica gel chromatography, 310 mg of 3, 389 mg of NH₄Cl and 469 mg of Zn were reacted to afford 4 (260 mg, 90.3%) as a light yellow solid. MS (ESI): mass calcd. for C₂₀H₂₁N₃O₂S₂ 399.53 m/z found 399.8 [M+H]⁺.
5. KOCN (121.5 mg, 1.5 mmol) was added in portions to a mixture of 4 (300 mg, 0.75 mmol) in AcOH/H₂O (4 mL/4 mL) and the mixture stirred for 18 h at room temperature. The mixture was concentrated and the residue was purified by Prep-HPLC to afford Example 004 (91.2 mg, 27.5% yield, 95.1% purity 214 nm) as a white solid. MS (ESI): mass calcd. for C₂₁H₂₂N₄O₃S₂ 442.55 m/z found 442.8 [M+H]⁺. ¹H NMR (400 MHz, DMSO-d6) δ ppm 8.45 (s, 1H), 7.87 (d, J=8.1, 2H), 7.52 (d, J=8.1, 2H), 7.26 (s, 1H), 7.18 (d, J=8.5, 1H), 7.05 (s, 1H), 7.02 (d, J=8.4, 1H), 5.82 (s, 2H), 4.48 (s, 2H), 4.13 (s, 2H), 3.63 (t, J=5.8, 2H), 3.19 (s, 3H), 2.80 (t, J=5.7, 2H).

### Example 005

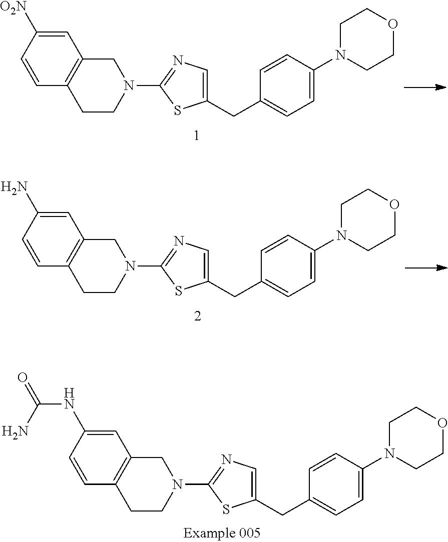 [SMILES: O=[N+]([O-])c1ccc2c(c1)CN(c1ncc(Cc3ccc(N4CCOCC4)cc3)s1)CC2 ; Nc1ccc2c(c1)CN(c1ncc(Cc3ccc(N4CCOCC4)cc3)s1)CC2 ; NC(=O)Nc1ccc2c(c1)CN(c1ncc(Cc3ccc(N4CCOCC4)cc3)s1)CC2] [0261] 1. Following the procedure described for Example 002, Step 3, 178 mg of 7-nitro-1,2,3,4-tetrahydroisoquinoline and 294 mg of Intermediate 3 were converted to 1 (300 mg) as a yellow solid.
2. Following the procedure described for Example 002, Step 4 except the reaction was heated at 50° C. for 3 h, 300 mg of 2, 74 mg of NH₄Cl and 90 mg of Zn were reacted to afford 2 (200 mg) as a yellow solid which was used without purification in the next step.
3. Following the procedure described for Example 001, Step 3, 200 mg of 2 and 81 mg of KCNO were reacted to afford Example 005 (25 mg, 11% yield) as a white solid; mass calcd. for C₂₄H₂₇N₅O₂S: 449.2; mass found: 449.8 [M+H]⁺. ¹H NMR (500 MHz, DMSO-d₆) δ 8.44 (s, 1H), 7.25 (s, 1H), 7.17 (d, J=8.1 Hz, 1H), 7.09 (d, J=8.1 Hz, 2H), 7.01 (d, J=8.2 Hz, 1H), 6.94 (s, 1H), 6.87 (d, J=8.2 Hz, 2H), 5.82 (s, 2H), 4.45 (s, 2H), 3.87 (s, 2H), 3.72 (s, 4H), 3.60 (t, J=5.5 Hz, 2H), 3.05 (s, 4H), 2.78 (s, 2H).

### Example 006

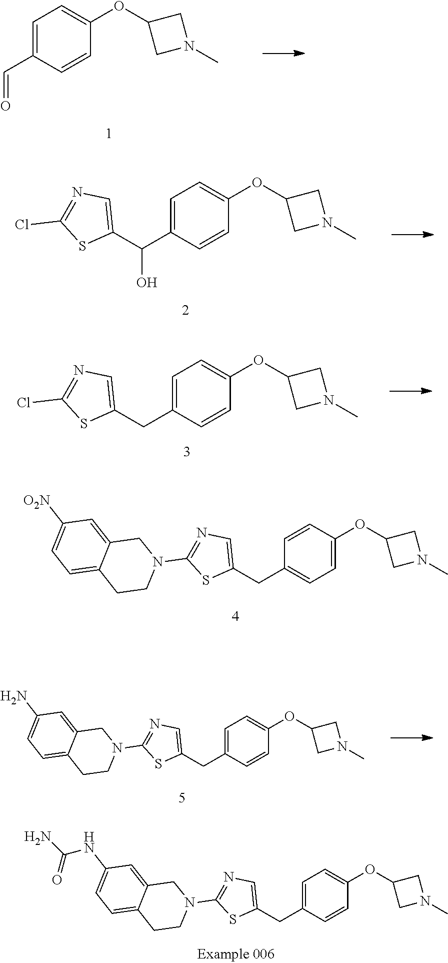 [SMILES: CN1CC(Oc2ccc(C=O)cc2)C1 ; CN1CC(Oc2ccc(C(O)c3cnc(Cl)s3)cc2)C1 ; CN1CC(Oc2ccc(Cc3cnc(Cl)s3)cc2)C1 ; CN1CC(Oc2ccc(Cc3cnc(N4CCc5ccc([N+](=O)[O-])cc5C4)s3)cc2)C1 ; CN1CC(Oc2ccc(Cc3cnc(N4CCc5ccc(N)cc5C4)s3)cc2)C1 ; CN1CC(Oc2ccc(Cc3cnc(N4CCc5ccc(NC(N)=O)cc5C4)s3)cc2)C1] [0265] 1. A mixture of 1-methylazetidin-3-ol hydrochloride (5.00 g, 40.46 mmol), 4-fluorobenzaldehyde (3.4 g, 26.97 mmol), t-BuOK (9 g, 80.91 mmol) in DMF (20 mL) was stirred at 120° C. in a sealed tube for 16 h. The mixture was diluted with sat. aq. NH₄Cl and extracted with EA. The combined extracts were washed with water, brine, and dried over Na₂SO₄, filtered and the filtrate concentrated afford 1 (2.6 g) as an oil, which was used directly in next step without further purification.
2. Following the procedure described for Example 002, Step 1, 1.95 g of 2-chlorothiazole and 2.6 g of 1 were converted to 2 (450 mg) which was obtained as a white solid.
3. Following the procedure described for Intermediate 3, Step 2, 450 mg of 2 was treated with 674 mg of TES to give 3 (300 mg) which was obtained as a white solid.
4. Following the procedure described for Example 002, Step 3, 200 mg of 3 and 120 mg of 7-nitro-1,2,3,4-tetrahydroisoquinoline were converted to 4 (230 mg) which was obtained as white solid.
5. Following the procedure described for Example 002, Step 4 except the mixture was heated at 70° C. for 4 h, 230 mg of 4, 140 mg of NH₄Cl and 172 mg of Zn were reacted to afford 5 (150 mg) as a yellow oil which was used directly in next step without further purification.
6. Following the procedure described for Example 001, Step 3, 150 mg of 5 and 60 mg of KCNO were reacted to afford Example 006 (20 mg, 12% yield) as a white solid. LCMS mass calcd. for C₂₄H₂₇N₅O₂S: 449.2; Mass Found: 450.2 [M+H]⁺. ¹H NMR (400 MHz, DMSO-d6) δ 8.42 (s, 1H), 7.25 (d, J=1.9 Hz, 1H), 7.17 (dd, J=8.2, 2.1 Hz, 1H), 7.13 (d, J=8.6 Hz, 2H), 7.01 (d, J=8.3 Hz, 1H), 6.94 (s, 1H), 6.73-6.75 (m, 2H), 5.80 (s, 2H), 4.68 (p, J=5.7 Hz, 1H), 4.45 (s, 2H), 3.90 (s, 2H), 3.65-3.77 (m, 2H), 3.60 (t, J=5.9 Hz, 2H), 2.85-2.97 (m, 2H), 2.79 (t, J=5.9 Hz, 2H).

### Example 007

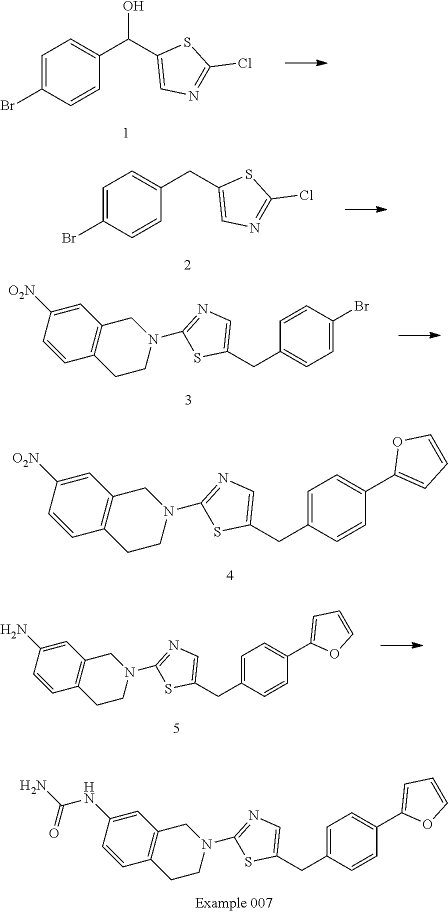 [SMILES: OC(c1ccc(Br)cc1)c1cnc(Cl)s1 ; Clc1ncc(Cc2ccc(Br)cc2)s1 ; O=[N+]([O-])c1ccc2c(c1)CN(c1ncc(Cc3ccc(Br)cc3)s1)CC2 ; O=[N+]([O-])c1ccc2c(c1)CN(c1ncc(Cc3ccc(-c4ccco4)cc3)s1)CC2 ; Nc1ccc2c(c1)CN(c1ncc(Cc3ccc(-c4ccco4)cc3)s1)CC2 ; NC(=O)Nc1ccc2c(c1)CN(c1ncc(Cc3ccc(-c4ccco4)cc3)s1)CC2] [0272] 1. To a solution of 2-chlorothiazole (5.76 g, 48 mmol) in dry THF (40 mL) at −78° C. under N₂ was added n-BuLi (2.4M, 20.0 mL, 48 mmol) dropwise. After 1 h, a solution of 4-bromobenzaldehyde (7.40 g, 40 mmol) in THF (40 mL) was added dropwise. The mixture was warmed to RT and stirred overnight. The mixture was diluted with sat. aq. NH₄Cl and extracted with EtOAc. The combined extracts were washed with brine, dried over Na₂SO₄, filtered, and the filtrate concentrated. The resulting residue was purified by chromatography on silica gel to give 1 (8.00 g, 66% yield) as a yellow oil. MS (ESI): mass calcd. for C₁₀H₇BrClNOS 304.59, m/z found 305.7 [M+H]⁺.
2. A mixture of 1 (8.00 g, 26.4 mmol) and TES (18 mL) in TFA (50 mL) was stirred at RT for 2 h, concentrated and the residue was diluted with sat. aq. NaHCO₃. The mixture was extracted with DCM and the combined organic extracts were washed with brine, dried over Na₂SO₄, filtered, the filtrate concentrated and the residue purified by chromatography on silica gel to give 2 (7.20 g, 94.7% yield) as a brown oil. MS (ESI): mass calcd. for C₁₀H₇BrClNS 288.59, m/z found 289.6 [M+H]⁺.
3. A mixture of 7-nitro-1,2,3,4-tetrahydroisoquinoline (200 mg, 1.07 mmol), 2 (401 mg, 1.39 mmol) and K₂CO₃ (444 mg, 3.21 mmol) in DMF (3 mL) was stirred at 120° C. for 4 h. The mixture was diluted with H₂O and extracted with EA. The combined extracts were washed with brine and dried over Na₂SO₄, filtered and the filtrate concentrated. The resulting residue was purified by silica gel column chromatography to give 3 (271 mg) as a yellow oil.
4. A mixture of 3 (270 mg, 0.63 mmol), furan-2-ylboronic acid (105 mg, 0.94 mmol), Pd(dppf)Cl₂ (46 mg, 0.06 mmol) and K₂CO₃ (260 mg, 1.88 mmol) in 1,4-dioxane (6 mL) and H₂O (1.5 mL) was stirred at 90° C. for 2 h. The mixture was diluted with H₂O and extracted with EA. The combined extracts were washed with brine, dried over Na₂SO₄ filtered and the filtrate concentrated. The resulting residue was purified by silica gel column chromatography to give 4 (120 mg) as a white solid.
5. A mixture of 4 (120 mg, 0.63 mmol), Zn (8.0 mg, 0.13 mmol), and NH₄Cl (7.0 mg, 0.13 mmol) in MeOH (1 mL) was stirred at 50° C. for 3 h. The mixture was filtered and the filtrate concentrated to give 5 (100 mg, 89.9%) as a white solid which was used without purification in the next step.
6. A mixture of 5 (100 mg, 0.26 mmol) and KCNO (52 mg, 0.52 mmol) in HOAc (2 mL) and H₂O (2 mL) was stirred at RT for 2 h. The mixture was diluted with NH₄HCO₃ (aq., 10 mL) and extracted with EA. The combined extracts were washed with brine and dried over Na₂SO₄, filtered and the filtrate concentrated. The resulting residue was purified by Prep-HPLC to give Example 007 (16.4 mg, 14.3% yield) as white solid after lyophylization. Mass calcd. for C₂₄H₂₂N₄O₂S: 430.2; mass found: 431.1 [M+H]⁺. ¹H NMR (400 MHz, DMSO-d6) δ 8.45 (s, 1H), 7.72 (d, J=1.2 Hz, 1H), 7.64 (d, J=8.8 Hz, 2H), 7.25-7.30 (m, 3H), 7.17 (dd, J=8.8, 2.4 Hz, 1H), 7.00-7.03 (m, 2H), 6.90 (d, J=3.6 Hz, 1H), 6.58 (dd, J=3.6, 1.2 Hz, 1H), 5.80 (s, 2H), 4.47 (s, 2H), 4.00 (s, 2H), 3.61 (t, J=5.9 Hz, 2H), 2.80 (t, J=5.6 Hz, 2H).

Example 008

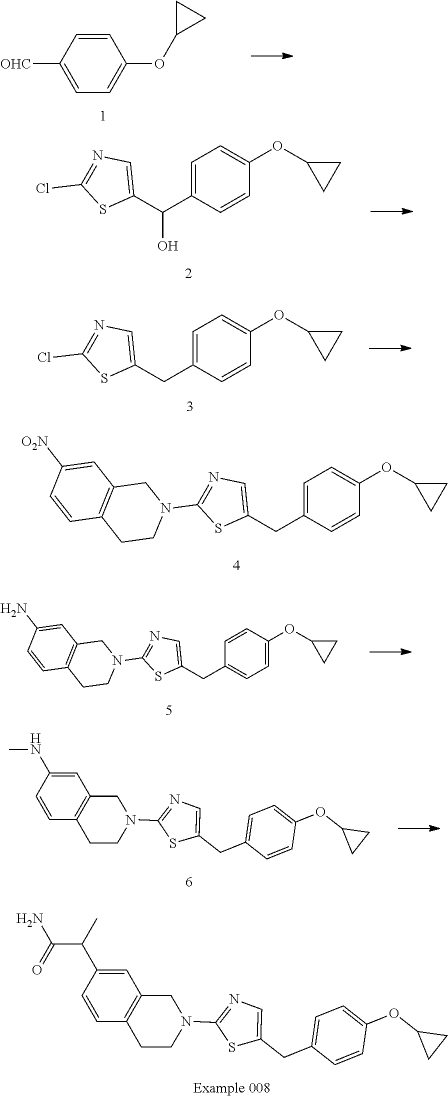

1. A mixture of 4-hydroxybenzaldehyde (2 g, 16.39 mmol), bromocyclopropane (19.8 g, 163 mmol), Cs₂CO₃ (13 g, 39.9 mmol), KI (264 mg, 1.59 mmol) in DMA (20 mL) was stirred at 120° C. for 72 h. The mixture was diluted with EA, washed with water, brine, dried over Na₂SO₄ filtered and the filtrate concentrated. The resulting residue was purified by silica gel chromatography to afford 1 (720 mg) as colorless oil.
2. Following the procedure described for Intermediate 1, Step 2 except 1.09 eq. of n-BuLi was used, 583 mg of 2-chlorothiazole and 720 mg of 1 were converted to 2 (1.2 g, oil) which was used directly in next step without further purification.
3. Following the procedure described for Intermediate 3, Step 2 except the combined EA extracts were washed with brine followed by drying over Na₂SO₄, 1.2 g of 2 was treated with 1.9 g of TES to give 3 (915 mg) as colorless oil.
4. Following the procedure described for Example 002, Step 3, 356 mg of 7-nitro-1,2,3,4-tetrahydroisoquinoline and 530 mg of 3 were converted to 4 (600 mg) obtained as a yellow solid.
5. Following the procedure described for Example 002, Step 4 except the reaction mixture was heated at 50° C. for 3 h, 600 mg of 4, 160 mg of NH₄Cl and 195 mg of Zn were reacted to afford 5 (450 mg) as a yellow solid which was used without purification in the next step.
6. To a solution of 5 (450 mg, 1.2 mmol) in DCM (10 mL) at RT was added NaBH(OAc)₃ (1.02 g, 4.8 mmol) and paraformaldehyde (54 mg) and the mixture stirred at 25° C. for 4 h. Water was added and the resulting mixture extracted with DCM. The combined extracts were washed with water, brine, dried over Na₂SO₄ filtered and the filtrate concentrated to give a residue which was purified by silica gel chromatography to give 6 (195 mg) as a yellow solid.
7. Following the procedure described for Example 001, Step 3, 195 mg of 6 and 81 mg of KCNO were reacted to afford Example 008 (35 mg, 16.1% yield) as a white solid. Mass calcd. for C₂₄H₂₆N₄O₂S: 434.2; mass found: 435.1 [M+H]⁺, ¹H NMR (400 MHz, DMSO) δ 7.17 (t, J=3.9 Hz, 2H), 7.11-7.15 (m, 2H), 7.07 (dd, J=8.1, 2.2 Hz, 1H), 6.98 (d, J=2.1 Hz, 1H), 6.96 (s, 2H), 5.73 (s, 2H), 4.51 (s, 2H), 3.91 (s, 2H), 3.76-3.80 (m, 1H), 3.63 (t, J=5.9 Hz, 2H), 3.10 (s, 3H), 2.87 (t, J=5.9 Hz, 2H), 0.72-0.78 (m, 2H), 0.59-0.64 (m, 2H).

### Example 009

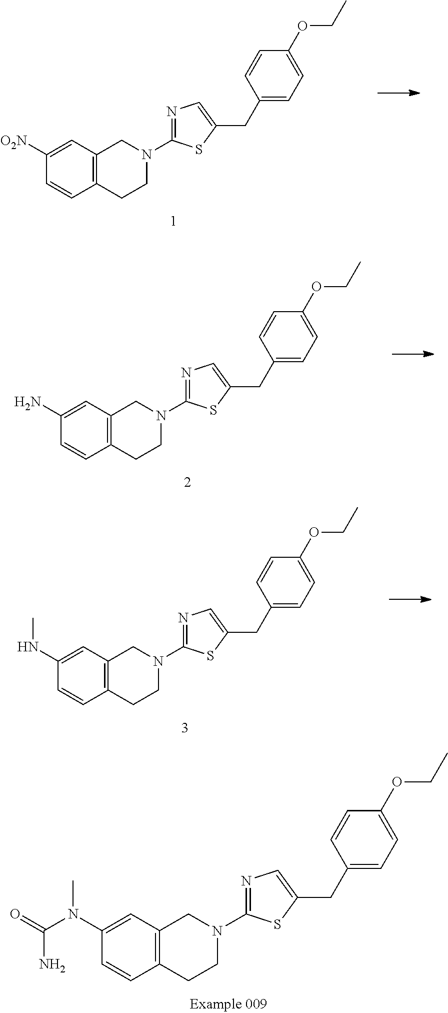 [0287] 1. Following the procedure described for Example 002, Step 3, 356 mg of 7-nitro-1,2,3,4-tetrahydroisoquinoline and 512 mg of Intermediate 4 were converted to 1 (590 mg) which was obtained as a yellow solid.
2. Following the procedure described for Example 002, Step 4, 590 mg of 1, 160 mg of NH₄Cl and 195 mg of Zn were reacted to give 2 (440 mg) as a yellow solid which was used without purification in the next step.
3. Following the procedure described for Example 008, Step 6, 440 mg of 1, 1.02 g of NaBH(OAc)₃ and 54 mg of paraformaldehyde were reacted to give 3 (190 mg) as a yellow solid.
4. Following the procedure described for Example 001, Step 3, 190 mg of 3 and 81 mg of KCNO were reacted to give Example 009 (30 mg, 14.2% yield) as a white solid. Mass calcd. for C₂₃H₂₆N₄O₂S: 422.2; mass found: 423.1 [M+H]⁺. ¹H NMR (400 MHz, DMSO) δ 7.17 (d, J=8.2 Hz, 1H), 7.14-7.16 (m, 1H), 7.13 (d, J=2.1 Hz, 2H), 7.08 (dd, J=8.1, 2.2 Hz, 1H), 6.95 (s, 1H), 6.82-6.87 (m, 2H), 5.73 (s, 2H), 4.52 (s, 2H), 3.98 (q, J=7.0 Hz, 2H), 3.91 (s, 2H), 3.63 (t, J=5.9 Hz, 2H), 3.11 (s, 3H), 2.87 (t, J=5.9 Hz, 2H), 1.31 (t, J=7.0 Hz, 3H).

### Example 010

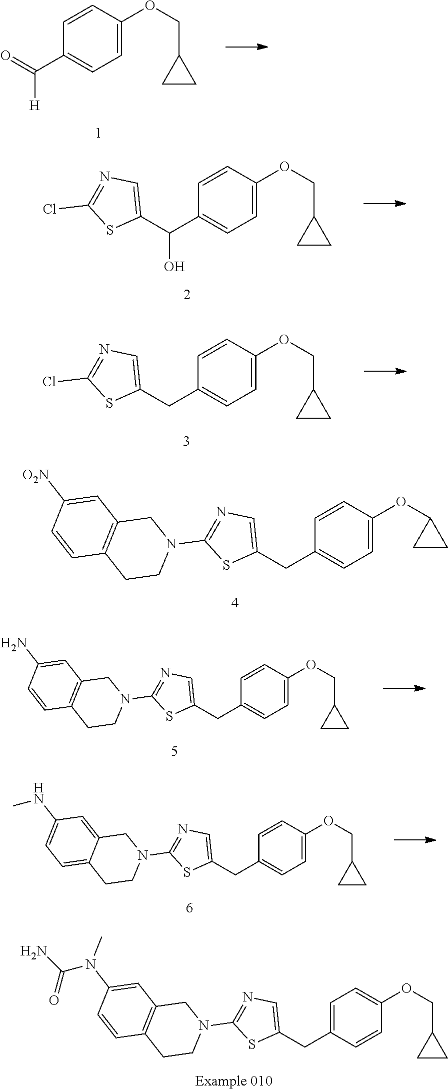 [0292] 1. A mixture of 4-hydroxybenzaldehyde (5 g, 41 mmol), (bromomethyl)cyclopropane (5.53 g, 41 mmol) and K₂CO₃ (14.1 g, 102.5 mmol) in DMF (100 mL) were stirred at RT overnight. The mixture was filtered and concentrated to give a residue which was purified by silica gel chromatography to afford 1 (5.8 g, 55% yield) as a white solid.
2. Following the procedure described for Example 002, Step 1 except the reaction mixture was stirred 5 h at RT after 1 h at −78° C., 3 g of 2-chlorothiazole and 4.5 g of 1 were converted to 2 (6.5 g, 86.7% yield) obtained as a white solid.
3. TES (10.2 g, 88 mmol) was added dropwise to a mixture of 2 (6.5 g, 22 mmol) in dry TFA (10 g, 88 mmol) and DCM (60 ml) at RT. After 2 h, the solution was concentrated, the residue treated with saturated NaHCO₃ and extracted with EA. The combined extracts were washed with brine, dried with Na₂SO₄, filtered and the filtrate concentrated to give a residue which was purified by silica gel chromatography to afford 3 (5 g, 76.9% yield) as a white solid.
4. Following the procedure described for Example 002, Step 3 except 3 eq. of Cs₂CO₃ was used and the mixture was heated at 100° C. for 12 h, 713 mg of 7-nitro-1,2,3,4-tetrahydroisoquinoline and 1.13 g of 3 were converted to 4 (1.05 g, 62.3%)
5. To a solution of 4 (1.05 g, 2.49 mmol) in MeOH (30 mL) was added Zn (488 mg, 7.47 mmol) and NH₄Cl (400 mg, (7.47 mmol). The resulting mixture was stirred at 80° C. for 4 h, filtered and the filtrate concentrated. The residue was diluted with H₂O, extracted with EA, the combined extracts dried over Na₂SO₄, filtered and the filtrate concentrated. The resulting residue was purified by a silica gel chromatography column to give 5 (790 mg).
6. A mixture of 5 (790 mg, 2.02 mmol), 37% formaldehyde (656 mg, 8.08 mmol), and NaBH₃CN (508 mg, 8.08 mmol) in DCM and MeOH was stirred at 40° C. for 12 h. The mixture was cooled and NaBH₄ (98 mg, 2.58 mmol) was added and the mixture heated at 40° C. for 4 h. The mixture was concentrated and diluted with H₂O. 1 N hydrochloric acid was added to adjust the pH to <5 then sat. NaHCO₃ was added until the pH 8. The resulting mixture was extracted with EA, the combined extracts were washed with brine, dried over Na₂SO₄, filtered and the filtrate concentrated. The resulting residue was purified by a silica gel chromatography column to give 6 (284 mg).
7. A mixture of 6 (284 mg, 0.70 mmol 1 eq), KCNO (114 mg, 1.40 mmol 2 eq) in HOAc (3 mL) and H₂O (3 mL) was stirred at 25° C. for 3 h. The mixture was diluted with H₂O, extracted with EA, the combined extracts were washed with brine, dried over Na₂SO₄, filtered and the filtrate concentrated. The resulting residue was purified by Prep-HPLC to give Example 010 (55.5 mg, 19% yield) as white solid after lyophylization. Mass calcd. for C25H28N4O2S: 448.2; mass found: 449.2 [M+H]⁺. ¹H NMR (400 MHz, DMSO-d6) δ 7.16 (d, J=8.8 Hz, 1H), 7.10-7.14 (m, 3H), 7.07 (dd, J=8.8, 1.2 Hz, 1H), 6.94 (s, 1H), 6.82-6.86 (m, 2H), 5.73 (s, 2H), 4.51 (s, 2H), 3.90 (s, 2H), 3.76 (d, J=8.8 Hz, 2H), 3.63 (t, J=5.6 Hz, 2H), 3.10 (s, 3H), 2.86 (t, J=5.9 Hz, 2H), 1.17-1.21 (m, 1H), 0.54-0.56 (m, 2H), 0.29-0.30 (m, 2H).

### Example 011

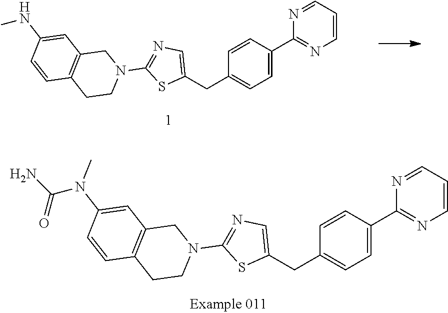 [SMILES: CNc1ccc2c(c1)CN(c1ncc(Cc3ccc(-c4ncccn4)cc3)s1)CC2 ; CN(C(N)=O)c1ccc2c(c1)CN(c1ncc(Cc3ccc(-c4ncccn4)cc3)s1)CC2] [0300] 1. Following the procedure described for Example 008, Step 6, 480 mg of Compound 2 from Example 001, 1.02 g of NaBH(OAc)3 and 54 mg of paraformaldehyde were reacted to give 1 (210 mg) as a yellow solid.
2. Following the procedure described for Example 001, Step 3, 210 mg of 1 and 81 mg of KCNO were reacted to afford Example 011 (35 mg, 15.4% yield) as a white solid. Mass calcd. for C₂₅H₂₄N₆OS: 456.2; mass found: 456.7 [M+H]⁺. ¹H NMR (400 MHz, DMSO) δ 8.89 (d, J=4.8 Hz, 2H), 8.33 (d, J=8.3 Hz, 2H), 7.42 (dd, J=12.6, 6.6 Hz, 3H), 7.15 (dd, J=16.2, 5.0 Hz, 2H), 7.07 (dd, J=8.1, 2.2 Hz, 1H), 7.04 (s, 1H), 5.73 (s, 2H), 4.53 (s, 2H), 4.08 (s, 2H), 3.64 (t, J=5.9 Hz, 2H), 3.10 (s, 3H), 2.87 (t, J=5.9 Hz, 2H).

### Example 012

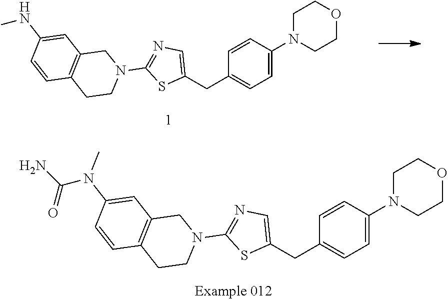 [SMILES: CNc1ccc2c(c1)CN(c1ncc(Cc3ccc(N4CCOCC4)cc3)s1)CC2 ; CN(C(N)=O)c1ccc2c(c1)CN(c1ncc(Cc3ccc(N4CCOCC4)cc3)s1)CC2] [0303] 1. Following the procedure described for Example 008, Step 6, 490 mg of Compound 2 from Example 005, 1.02 g of NaBH(OAc)₃ and 54 mg of paraformaldehyde were reacted to give 1 (210 mg) as a yellow solid.
2. Following the procedure described for Example 001, Step 3, 210 mg of 1 and 81 mg of KCNO were reacted to afford Example 012 (35 mg, 15.1% yield) as a white solid. Mass calcd. for C₂₅H₂₉NO₂S: 463.2; mass found: 463.3 [M+H]⁺. ¹H NMR (400 MHz, DMSO) δ 7.17 (d, J=8.1 Hz, 1H), 7.12 (d, J=8.3 Hz, 2H), 7.08 (dd, J=7.9, 2.1 Hz, 2H), 6.95 (s, 1H), 6.88 (d, J=8.7 Hz, 2H), 5.73 (s, 2H), 4.52 (s, 2H), 3.88 (s, 2H), 3.70-3.75 (m, 4H), 3.63 (t, J=5.9 Hz, 2H), 3.11 (s, 3H), 3.03-3.08 (m, 4H), 2.87 (t, J=5.8 Hz, 2H).

### Example 013

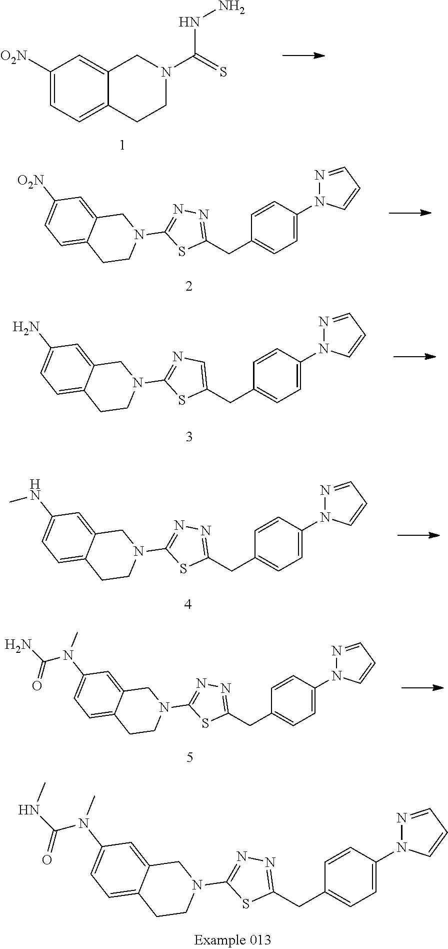 [SMILES: NNC(=S)N1CCc2ccc([N+](=O)[O-])cc2C1 ; O=[N+]([O-])c1ccc2c(c1)CN(c1nnc(Cc3ccc(-n4cccn4)cc3)s1)CC2 ; Nc1ccc2c(c1)CN(c1ncc(Cc3ccc(-n4cccn4)cc3)s1)CC2 ; CNc1ccc2c(c1)CN(c1nnc(Cc3ccc(-n4cccn4)cc3)s1)CC2 ; CN(C(N)=O)c1ccc2c(c1)CN(c1nnc(Cc3ccc(-n4cccn4)cc3)s1)CC2 ; CNC(=O)N(C)c1ccc2c(c1)CN(c1nnc(Cc3ccc(-n4cccn4)cc3)s1)CC2] [0306] 1. A mixture of 7-nitro-1,2,3,4-tetrahydroisoquinoline (2.0 g, 11.22 mmol), 1,1′-thiocarbonyldiimidazole (2.0 g, 11.22 mmol) and K₂CO₃ (775 mg, 5.61 mmol) in DMF (20 mL) was stirred at 80° C. for 1 h and NH₂NH₂·H₂O (1.1 g, 22.44 mmol) was added. The mixture was stirred at 80° C. for 1 h, cooled, poured into water and the resulting mixture extracted with EtOAc. The combined organic extracts were washed with water, dried over Na₂SO₄, filtered and the filtrate concentrated to afford 1 (2.3 g) as brown solid which was used directly in next step.
2. A mixture of 1 (1 g, 3.96 mmol), Intermediate 5 (680 mg, 3.37 mmol) and POCl₃ (3.0 mL) was stirred at 75° C. for 1 h, the mixture was cooled and H₂O added. The resulting mixture was stirred at 110° C. for 1 h. The mixture was cooled, poured into water and adjusted to pH 9˜10 with aq. Na₂CO₃. The resulting precipitate was collected by filtration and washed with H₂O to afford 2 (1.2 g) as a yellow solid, which was used directly in next step.
3. Following the procedure described for Example 002, Step 4 except the reaction mixture was stirred at 70° C. for 4 h, 1.2 g of 2, 764 mg of NH₄Cl and 935 mg of Zn were reacted to afford 3 (900 mg) which was used in next step without further purification.
4. Following the procedure described for Example 008, Step 6 except the reaction mixture was diluted with sat. NaHCO₃ after 4 h at 25° C., 360 mg of 3, 982 mg of NaBH(OAc)₃ and 33 mg of paraformaldehyde were reacted to give 4 (500 mg) as white solid which was used in next step without purification.
5. Following the procedure described for Example 001, Step 3, 400 mg of 4 and 82 mg of KCNO were reacted in HOAc (2 mL) and H₂O (2 mL) to afford 5 (300 mg) as a white solid.
6. To a solution of 5 (230 mg, 0.516 mmol) in THF at 0° C. (5 mL) was added NaH (15 mg, 0.619 mmol) and the mixture stirred at 0° C. for 2 h. MeI (80 mg, 0.567 mmol) was added and the mixture warmed to room temperature and stirred overnight. The mixture was diluted with aq. NaHCO₃ and extracted with EA, the combined extracts washed brine and dried over Na₂SO₄. The mixture was filtered and the filtrate concentrated to give a residue which was purified by Prep-HPLC to give Example 013 (8 mg, 3.4% yield) as white solid after lyophylization. Mass calcd. for C₂₄H₂₅N₇OS: 459.2; Mass Found: 459.8 [M+H]⁺. ¹H NMR (400 MHz, DMSO-d6) δ 8.47 (d, J=2.4 Hz, 1H), 7.81 (d, J=8.6 Hz, 2H), 7.73 (d, J=1.6 Hz, 1H), 7.43 (d, J=8.6 Hz, 2H), 7.19 (d, J=8.1 Hz, 1H), 7.12 (d, J=1.9 Hz, 1H), 7.06 (dd, J=8.1, 2.2 Hz, 1H), 6.50-6.56 (m, 1H), 5.83 (s, 1H), 4.59 (s, 2H), 4.29 (s, 2H), 3.69 (t, J=6.0 Hz, 2H), 3.10 (s, 3H), 2.90 (t, J=5.9 Hz, 2H), 2.53 (d, J=2.8 Hz, 3H).

### Example 014

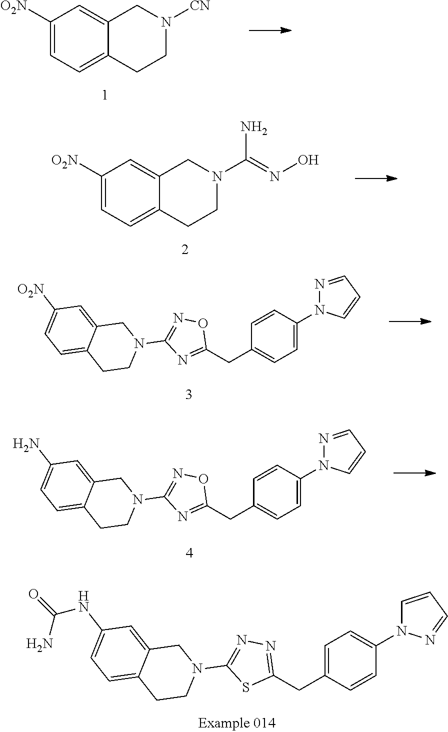 [SMILES: N#CN1CCc2ccc([N+](=O)[O-])cc2C1 ; N/C(=N\O)N1CCc2ccc([N+](=O)[O-])cc2C1 ; O=[N+]([O-])c1ccc2c(c1)CN(c1noc(Cc3ccc(-n4cccn4)cc3)n1)CC2 ; Nc1ccc2c(c1)CN(c1noc(Cc3ccc(-n4cccn4)cc3)n1)CC2 ; NC(=O)Nc1ccc2c(c1)CN(c1nnc(Cc3ccc(-n4cccn4)cc3)s1)CC2] [0313] 1. A mixture of 7-nitro-1,2,3,4-tetrahydroisoquinoline (2 g, 11.24 mmol), CNBr (1.4 g, 13.49 mmol), K₂CO₃ (2.3 g, 16.86 mmol) in acetone (20 mL) was stirred at room temperature for 1 h. The solvent was removed and the residue diluted with EA and the mixture washed with brine, dried over Na₂SO₄, filtered and the filtrate concentrated. The residue was purified by silica gel column chromatography to afford 1 (540 mg) as yellow solid.
2. TEA (276 mg, 2.74 mmol) and NH₂OH·HCl (189 mg, 2.74 mmol) were added to a solution of 1 (530 mg, 2.61 mmol) in EtOH (10 mL) and the mixture was stirred at 80° C. for 30 min. The mixture was concentrated to afford 2 (900 mg) which was used directly in next step without further purification.
3. To a suspension of Intermediate 5 (590 mg, 2.96 mmol) in DCM (10 mL) was added CDI (476 mg, 2.96 mmol) and the mixture was stirred at 55° C. for 1 h and 2 (700 mg, 2.96 mmol) was added. The resulting mixture was stirred at 55° C. for 1 h, the mixture was cooled to RT and the solvent was removed. Toluene (10 mL) was added to the residue and the mixture was heated at 150° C. under microwave for 40 min. The mixture was concentrated and the residue was purified by silica gel column chromatography to afford 3 (470 mg) as yellow solid.
4. Following the procedure described for Example 002, Step 4 except the reaction mixture was heated at 70° C. for 4 h, 100 mg of 3, 132 mg of NH₄Cl and 162 mg of Zn were reacted to afford 4 (220 mg) which was used directly in next step without purification.
5. Following the procedure described for Example 001, Step 3, 220 mg of 4 and 95 mg of KCNO in HOAc (2 mL) and water (2 mL) Example 014 (30 mg, 12.2% yield) as a white solid. Mass calcd. for C₂₂H₂₁N₇O₂: 415.2; Mass Found: 416.2 [M+H]⁺. ¹H NMR (400 MHz, DMSO-d6) δ 8.48 (d, J=2.5 Hz, 1H), 8.41 (s, 1H), 7.80-7.85 (m, 2H), 7.74 (d, J=1.5 Hz, 1H), 7.46 (d, J=8.6 Hz, 2H), 7.24 (d, J=1.9 Hz, 1H), 7.17 (dd, J=8.3, 2.2 Hz, 1H), 7.00 (d, J=8.3 Hz, 1H), 6.54 (dd, J=2.4, 1.8 Hz, 1H), 5.79 (s, 2H), 4.44 (s, 2H), 4.26 (s, 2H), 3.62 (dd, J=8.1, 3.8 Hz, 2H), 2.77 (t, J=5.9 Hz, 2H).

### Example 015

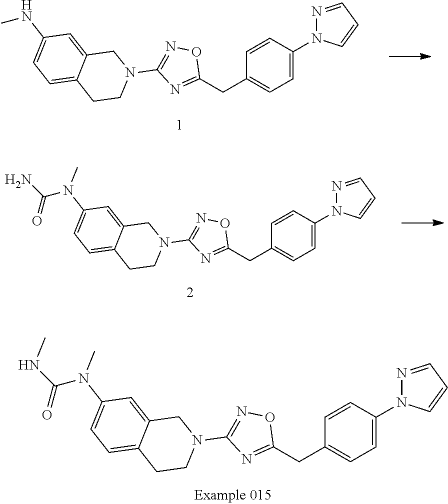 [SMILES: CNc1ccc2c(c1)CN(c1noc(Cc3ccc(-n4cccn4)cc3)n1)CC2 ; CN(C(N)=O)c1ccc2c(c1)CN(c1noc(Cc3ccc(-n4cccn4)cc3)n1)CC2 ; CNC(=O)N(C)c1ccc2c(c1)CN(c1noc(Cc3ccc(-n4cccn4)cc3)n1)CC2] [0319] 1. A mixture of Compound 4 from Example 014 (370 mg, 0.995 mmol), paraformaldehyde (30 mg), NaBH(OAc)₃ (840 mg, 3.98 mmol) in DCM (15 mL) was stirred at room temperature for 3 h and the mixture diluted saturated aq. NaHCO3 solution. The mixture was extracted with DCM, the combined extracts washed with brine and concentrated. The residue was purified to afford 1 (290 mg) as white solid.
2. Following the procedure described for Example 001, Step 3, 290 mg of 1 and 123 mg of KCNO were reacted to afford 2 (100 mg) as a white solid.
3. Following the procedure described for Example 013, Step 6, 100 mg of 2, 7 mg of NaH and 36 mg of MeI were reacted to afford Example 015 (3.5 mg, 3.4% yield) as white solid after lyophylization. Mass calcd. for C₂₄H₂₅N₇O₂: 443.2; mass Found: 443.8 [M+H]⁺. ¹H NMR (400 MHz, CDCl₃) δ 7.90 (d, J=1.7 Hz, 1H), 7.69 (dd, J=21.8, 6.9 Hz, 3H), 7.43 (d, J=8.4 Hz, 2H), 7.20 (d, J=7.9 Hz, 1H), 7.01-7.14 (m, 2H), 6.46 (s, 1H), 4.60 (s, 2H), 4.37 (s, 2H), 4.27-4.34 (m, 1H), 3.77 (t, J=5.7 Hz, 2H), 3.24 (s, 3H), 2.94 (t, J=5.4 Hz, 2H), 1.75 (d, J=7.2 Hz, 3H).

### Example 016

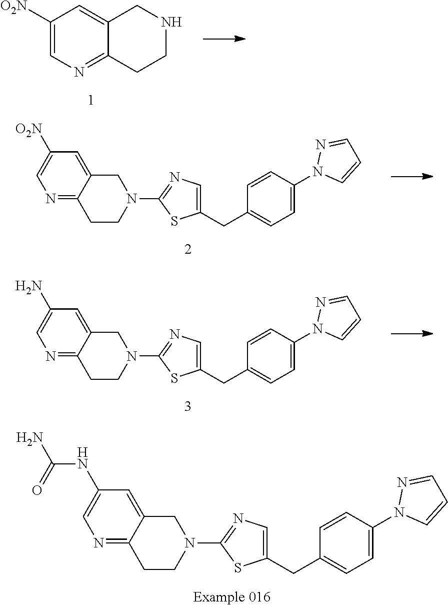 [SMILES: O=[N+]([O-])c1cnc2c(c1)CNCC2 ; O=[N+]([O-])c1cnc2c(c1)CN(c1ncc(Cc3ccc(-n4cccn4)cc3)s1)CC2 ; Nc1cnc2c(c1)CN(c1ncc(Cc3ccc(-n4cccn4)cc3)s1)CC2 ; NC(=O)Nc1cnc2c(c1)CN(c1ncc(Cc3ccc(-n4cccn4)cc3)s1)CC2] [0323] 1. To a solution of tert-butyl 3-nitro-7,8-dihydro-5H-1,6-naphthyridine-6-carboxylate (WO-2019200120-A1, 2 g, 7.16 mmol) in DCM (15 mL) was added TFA (5 mL) and the mixture was stirred at RT for 4 h. The mixture was concentrated to give 1 (1.5 g) which was used without purification in the next step.
2. A mixture of 1 (300 mg, 1.39 mmol), Intermediate 6 (383 mg, 1.39 mmol), Pd₂(dba)₃ (190 mg, 0.21 mmol), S-Phos (171 mg, 0.42 mmol) and Cs₂CO₃ (1.81 g, 5.56 mmol) in 1,4-dioxane (10 mL) was stirred at reflux for 4 h. The mixture was diluted with H₂O, extracted with EA, the combined extracts were washed with brine, dried over Na₂SO₄, filtered and the filtrate concentrated. The resulting residue was purified by silica gel chromatography column to give 2 (106 mg).
3. Following the procedure described for Example 002, Step 4 except the reaction mixture was heated at 70° C. for 16 h, 106 mg of 2, 64.3 mg of NH₄Cl and 78.2 mg of Zn were reacted to afford 3 (91 mg) which was used without purification in the next step.
4. A mixture of 3 (91 mg, 0.23 mmol), KCNO (38 mg, 0.46 mmol) in THF (1 mL), HOAc (0.5 mL) and H₂O (0.5 mL) was stirred at 25° C. for 3 h. The mixture was diluted with H₂O and extracted with EA. The combined extracts were washed with brine, dried over Na₂SO₄, filtered and the filtrate concentrated. The resulting residue was purified by Prep-HPLC to give Example 016 (26.04 mg, 27% yield) as white solid after lyophylization. Mass calcd. for C22H21N7OS: 431.2; mass found: 431.8 [M+H]⁺. ¹H NMR (400 MHz, DMSO-d6) δ 8.64 (s, 1H), 8.45 (d, J=1.6 Hz, 1H), 8.32 (d, J=1.6 Hz, 1H), 7.72-7.77 (m, 3H), 7.36 (d, J=12.0 Hz, 2H), 7.02 (s, 1H), 6.52 (t, J=2.1 Hz, 2H), 5.96 (s, 2H), 4.52 (s, 2H), 4.04 (s, 2H), 3.73 (t, J=5.9 Hz, 2H), 2.89 (t, J=5.6 Hz, 2H).

### Example 017

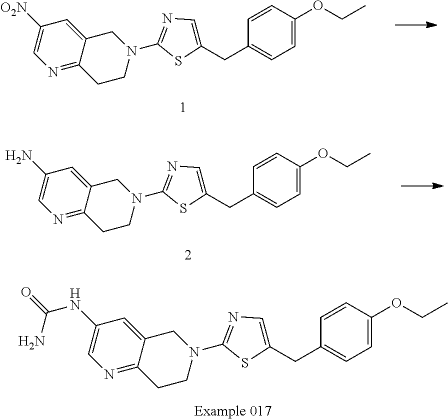 [SMILES: CCOc1ccc(Cc2cnc(N3CCc4ncc([N+](=O)[O-])cc4C3)s2)cc1 ; CCOc1ccc(Cc2cnc(N3CCc4ncc(N)cc4C3)s2)cc1 ; CCOc1ccc(Cc2cnc(N3CCc4ncc(NC(N)=O)cc4C3)s2)cc1] [0328] 1. A mixture of Compound 1 from Example 016 (230 mg, 1.29 mmol), Intermediate 4 (322 mg, 1.29 mmol), Pd₂(dba)₃ (176.7 mg, 0.193 mmol), S-Phos (158.2 mg, 0.386 mmol) and Cs₂CO₃ (837.2 mg, 2.58 mmol) in toluene (4 mL) was stirred at 140° C. in a microwave for 40 min. The mixture was diluted with H₂O and extracted with EA. The combined organic extracts were washed with brine, dried over Na₂SO₄, filtered and the filtrate concentrated. The resulting residue was purified by a silica gel chromatography column to give 1 (121 mg).
2. Following the procedure described for Example 002, Step 4 except the reaction mixture was heated at 70° C. for 16 h, 121 mg of 1, 97 mg of NH₄Cl and 118 mg of Zn were reacted to afford 2 (120 mg) as yellow solid which was used without purification in the next step.
3. Following the procedure described for Example 016, Step 4 except the reagents were mixed in THF (0.5 mL), HOAc (1 mL) and H₂O (1 mL), 120 mg of 2 and 79 mg of KCNO were reacted to afford Example 017 (25.2 mg, 19.4% yield) as white solid after lyophylization. Mass calcd. for C21H23N5O2S: 409.2; mass found: 410.1 [M+H]⁺. ¹H NMR (400 MHz, DMSO-d6) δ 8.68 (s, 1H), 8.33 (d, J=1.2 Hz, 1H), 7.73 (d, J=1.2 Hz, 1H), 7.13 (d, J=8.0 Hz, 2H), 6.95 (s, 1H), 6.84 (d, J=8.0 Hz, 2H), 5.98 (s, 2H), 4.51 (s, 2H), 3.97 (dd, J=12.8, 3.6 Hz, 2H), 3.91 (s, 2H), 3.72 (t, J=5.9 Hz, 2H), 2.88 (t, J=5.6 Hz, 2H), 1.30 (t, J=9.5 Hz, 3H).

### Example 018

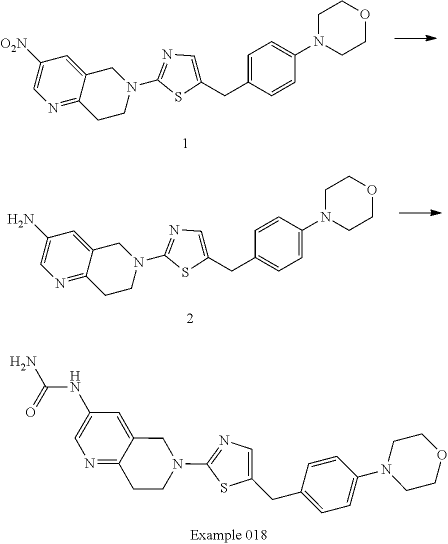 [SMILES: O=[N+]([O-])c1cnc2c(c1)CN(c1ncc(Cc3ccc(N4CCOCC4)cc3)s1)CC2 ; Nc1cnc2c(c1)CN(c1ncc(Cc3ccc(N4CCOCC4)cc3)s1)CC2 ; NC(=O)Nc1cnc2c(c1)CN(c1ncc(Cc3ccc(N4CCOCC4)cc3)s1)CC2] [0332] 1. A mixture of Compound 1 from Example 016 (145 mg, 0.84 mmol), Intermediate 3 (248 mg, 0.84 mmol), Pd₂(dba)₃ (114 mg, 0.125 mmol 0.15 eq), S-Phos (102.5 mg, 0.25 mmol 0.3) and Cs₂CO₃ (546 mg, 1.68 mmol 2 eq) in toluene (3 mL) was stirred at 110° C. for 2 h. The mixture was diluted with H₂O, extracted with EA, the combined extracts were washed with brine, dried over Na₂SO₄ and filtered. The filtrate was concentrated and the residue purified by a silica gel chromatography column to give 1 (113 mg).
2. Following the procedure described for Example 002, Step 4 except the mixture was heated at 70° C. for 16 h, 113 mg of 1, 64 mg of NH₄Cl and 78 mg of Zn were reacted to afford 2 (92 mg) which was used without purification in the next step.
3. Following the procedure described for Example 016, Step 4, 92 mg of 2 and 43 mg of KCNO were reacted to afford Example 018 (30.3 mg, 30% yield) as off-white solid after lyophylization. Mass calcd. for C23H26N6O2S: 450.2; mass found: 448.9 [M−H]⁻. ¹H NMR (400 MHz, DMSO-d6) δ 8.64 (s, 1H), 8.32 (d, J=1.2 Hz, 1H), 7.73 (s, 1H), 7.09 (d, J=8.8 Hz, 2H), 6.95 (s, 1H), 6.87 (d, J=8.8 Hz, 2H), 5.96 (s, 1H), 4.51 (s, 2H), 3.88 (s, 2H), 3.72 (t, J=5.9 Hz, 6H), 3.05 (t, J=3.2 Hz, 4H), 2.88 (t, J=5.6 Hz, 3H).

### Example 019

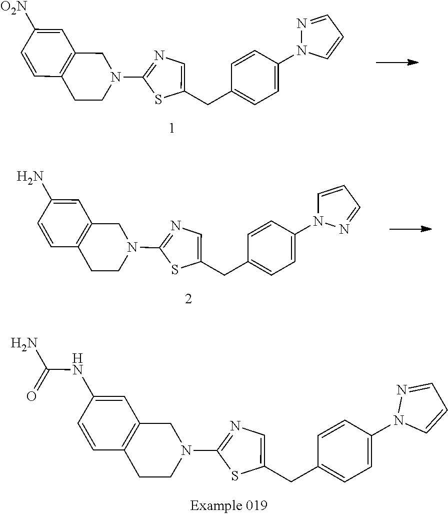 [SMILES: O=[N+]([O-])c1ccc2c(c1)CN(c1ncc(Cc3ccc(-n4cccn4)cc3)s1)CC2 ; Nc1ccc2c(c1)CN(c1ncc(Cc3ccc(-n4cccn4)cc3)s1)CC2 ; NC(=O)Nc1ccc2c(c1)CN(c1ncc(Cc3ccc(-n4cccn4)cc3)s1)CC2] [0336] 1. Following the procedure described for Example 002, Step 3, 1.78 g of 7-nitro-1,2,3,4-tetrahydroisoquinoline and 2.75 g of Intermediate 6 were converted to 1 (2.2 g) which was obtained as a yellow solid.
2. Following the procedure described for Example 002, Step 4 except the reaction mixture was stirred at RT for 3 h, 2.2 g of 1, 562 mg of NH₄Cl and 676 mg of Zn were reacted to afford 2 (1.55 g) as a yellow solid after purification by Combi-Flash chromatography.
3. Following the procedure described for Example 001, Step 3, 1.55 g of 2 and 648 mg of KCNO in HOAc (8 mL) and H₂O (8 mL) were reacted to afford Example 019 (610 mg, 35.5% yield) as a white solid. Mass calcd. for C₂₃H₂₂N₆OS: 430.2; mass found: 430.8 [M+H]⁺. ¹H NMR (400 MHz, DMSO) δ 8.43-8.45 (m, 2H), 7.71-7.78 (m, 3H), 7.35 (d, J=8.6 Hz, 2H), 7.25 (d, J=2.0 Hz, 1H), 7.17 (dd, J=8.2, 2.2 Hz, 1H), 7.01 (t, J=4.1 Hz, 2H), 6.52 (dd, J=2.4, 1.8 Hz, 1H), 5.79 (s, 2H), 4.47 (s, 2H), 4.03 (s, 2H), 3.61 (t, J=5.9 Hz, 2H), 2.79 (t, J=5.9 Hz, 2H).

### Example 020

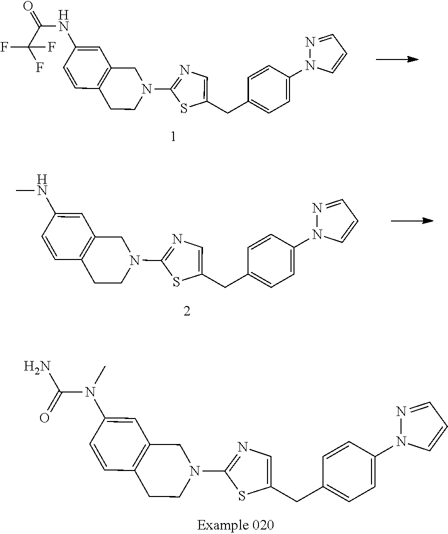 [SMILES: O=C(Nc1ccc2c(c1)CN(c1ncc(Cc3ccc(-n4cccn4)cc3)s1)CC2)C(F)(F)F ; CNc1ccc2c(c1)CN(c1ncc(Cc3ccc(-n4cccn4)cc3)s1)CC2 ; CN(C(N)=O)c1ccc2c(c1)CN(c1ncc(Cc3ccc(-n4cccn4)cc3)s1)CC2] [0340] 1. To a solution of Compound 2 from Example 019 (387 mg, 1 mmol), TEA (303 mg, 3 mmol) in DCM (10 mL), TFAA (252 mg, 1.2 mmol) was added at 0° C., the mixture was warmed to RT and stirred at 25° C. for 2 h. The solution was diluted with EA, washed with water, brine and dried over Na₂SO₄, filtered and the filtrate concentrated 1 (510 mg, 99% yield) as a yellow solid which was used without purification in the next step. MS (ESI): mass calcd. for C₂₄H₂₀F₃N₅OS 483.5, m/z found 484 [M+H]⁺.
2. To a mixture of 1 (500 mg, 1 mmol), K₂CO₃ (276 mg, 2 mmol) in MeCN (10 mL), MeI (284 mg, 2 mmol) was added at 0° C., the mixture was warmed to RT and stirred at 25° C. for 6 hours. The mixture was diluted with EA, washed with water, brine, dried over Na₂SO₄, filtered, and the filtrate concentrated. The resulting residue was diluted with MeOH (10 mL), K₂CO₃ (276 mg, 2 mmol) was added and the mixture stirred at RT for 2 h. The mixture was diluted with EA, washed with water, brine, dried over Na₂SO₄, filtered, and the filtrate concentrated. The residue was purified by Combi-Flash chromatography to give 2 (280 mg, 71% yield) as a yellow solid. MS (ESI): mass calcd. for C₂₃H₂₃N₅S 401.5, m/z found 402 [M+H]⁺.
3. Following the procedure described for Example 001, Step 3, 100 mg of 2 and 41 mg of KCNO were reacted to afford Example 020 (20 mg, 9% yield) as a white solid. MS (ESI): mass calcd. for C₂₄H₂₄N₆OS 444.6, m/z found 445 [M+H]⁺. ¹H NMR (400 MHz, DMSO-d6) δ ppm 8.46 (s, 1H), 7.77 (d, J=8.1 Hz, 2H), 7.73 (s, 1H), 7.36 (d, J=8.1 Hz, 2H), 7.17 (d, J=8.1 Hz, 1H), 7.13 (s, 1H), 7.08 (d, J=7.9 Hz, 1H), 7.02 (s, 1H), 6.53 (s, 1H), 5.76 (s, 2H), 4.53 (s, 2H), 4.04 (s, 2H), 3.64 (t, J=5.7 Hz, 2H), 3.10 (s, 3H), 2.88 (t, J=5.3 Hz, 2H).

### Example 021

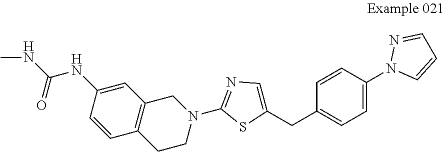 [SMILES: CNC(=O)Nc1ccc2c(c1)CN(c1ncc(Cc3ccc(-n4cccn4)cc3)s1)CC2] [0344] 1. A solution of Compound 2 from Example 019 (98 mg, 0.25 mmol), 4-nitrophenyl chloroformate (50 mg, 0.25 mmol), TEA (50 mg, 0.5 mmol) in DCM (10 mL) was stirred at 45° C. for 16 h. The mixture was cooled to 0° C. and a solution of methylamine hydrochloride (67 mg, 1 mmol), DMAP (5 mg), TEA (152 mg, 1.5 mmol) in DCM (10 mL) was added. the mixture was warmed to RT and stirred at 25° C. for 3 h, diluted with EA, washed with water, brine and dried over Na₂SO₄, filtered, and the filtrate concentrated. The resulting residue was purified by Prep-HPLC to give Example 021 (25 mg, 24% yield) as a white solid. MS (ESI): mass calcd. for C₂₄H₂₄N₆OS 444.6, m/z found 445 [M+H]⁺. ¹H NMR (400 MHz, DMSO-d6) δ ppm 8.49-8.42 (m, 2H), 7.77 (d, J=8.3 Hz, 2H), 7.73 (s, 1H), 7.36 (d, J=8.3 Hz, 2H), 7.24 (s, 1H), 7.19 (d, J=8.7 Hz, 1H), 7.01 (d, J=6.0 Hz, 2H), 6.53 (s, 1H), 6.00 (d, J=4.4 Hz, 1H), 4.47 (s, 2H), 4.04 (s, 2H), 3.62 (t, J=5.8 Hz, 2H), 2.80 (t, J=5.7 Hz, 2H), 2.63 (d, J=4.5 Hz, 3H).

### Example 022

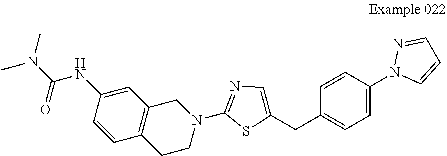 [SMILES: CN(C)C(=O)Nc1ccc2c(c1)CN(c1ncc(Cc3ccc(-n4cccn4)cc3)s1)CC2] [0346] 1. Following the procedure described for Example 021, Compound 2 from Example 019 (98 mg, 0.25 mmol) was reacted with 4-nitrophenyl chloroformate (50 mg, 0.25 mmol) followed by dimethylamine hydrochloride (82 mg, 1 mmol) to afford Example 022 (25 mg, 24% yield) as a white solid. MS (ESI): mass calcd. for C₂₅H₂₆N₆OS 458.6, m/z found 459 [M+H]⁺. ¹H NMR (400 MHz, DMSO-d6) δ ppm 8.45 (s, 1H), 8.22 (s, 1H), 7.77 (d, J=8.2 Hz, 2H), 7.72 (s, 1H), 7.36 (d, J=8.2 Hz, 2H), 7.31 (s, 1H), 7.26 (d, J=8.5 Hz, 1H), 7.03 (d, J=6.9 Hz, 2H), 6.53 (s, 1H), 4.47 (s, 2H), 4.04 (s, 2H), 3.62 (t, J=5.8 Hz, 2H), 2.91 (s, 6H), 2.81 (t, J=5.6 Hz, 2H).

### Example 023

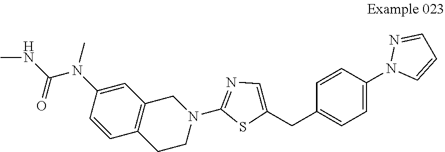 [SMILES: CNC(=O)N(C)c1ccc2c(c1)CN(c1ncc(Cc3ccc(-n4cccn4)cc3)s1)CC2] [0348] 1. Following the procedure described for Example 021, Compound 2 from Example 020 (100 mg, 0.25 mmol) was reacted with 4-nitrophenyl chloroformate (50 mg, 0.25 mmol) followed by methylamine hydrochloride (67 mg, 1 mmol) to afford Example 023 (20 mg, 23% yield) as a white solid. MS (ESI): mass calcd. for C₂₅H₂₆N₆OS 458.6, m/z found 459 [M+H]⁺. ¹H NMR (400 MHz, DMSO-d6) δ ppm 8.46 (s, 1H), 7.77 (d, J=8.2 Hz, 2H), 7.73 (s, 1H), 7.36 (d, J=8.2 Hz, 2H), 7.18 (d, J=8.0 Hz, 1H), 7.11 (s, 1H), 7.09-7.01 (m, 2H), 6.53 (s, 1H), 5.83 (d, J=4.4 Hz, 1H), 4.53 (s, 2H), 4.04 (s, 2H), 3.65 (t, J=5.8 Hz, 2H), 3.10 (s, 3H), 2.88 (t, J=5.8 Hz, 2H), 2.53 (s, 3H).

### Example 024

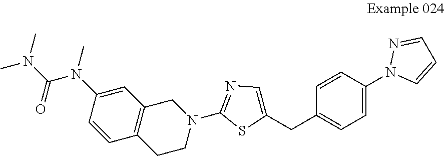 [SMILES: CN(C)C(=O)N(C)c1ccc2c(c1)CN(c1ncc(Cc3ccc(-n4cccn4)cc3)s1)CC2] [0350] 1. Following the procedure described for Example 021, Compound 2 from Example 020 (100 mg, 0.25 mmol) was reacted with 4-nitrophenyl chloroformate (50 mg, 0.25 mmol) followed by dimethylamine hydrochloride (82 mg, 1 mmol) to afford Example 024 (14 mg, 16% yield) as a white solid. MS (ESI): mass calcd. for C₂₆H₂₈N₆OS, m/z found 473 [M+H]⁺. ¹H NMR (400 MHz, DMSO-d6) δ ppm 8.16 (s, 1H), 7.31 (d, J=8.4 Hz, 2H), 7.22 (d, J=8.3 Hz, 1H), 7.12 (d, J=8.4 Hz, 2H), 6.98 (s, 2H), 6.96 (s, 1H), 6.92 (s, 1H), 4.59 (s, 2H), 3.90 (s, 2H), 3.73 (s, 4H), 3.67 (t, J=5.7 Hz, 2H), 3.11 (s, 4H), 2.94 (t, J=5.5 Hz, 2H), 2.23 (s, 3H), 2.08 (s, 3H).

### MCF7 Cell Growth Inhibition Assay

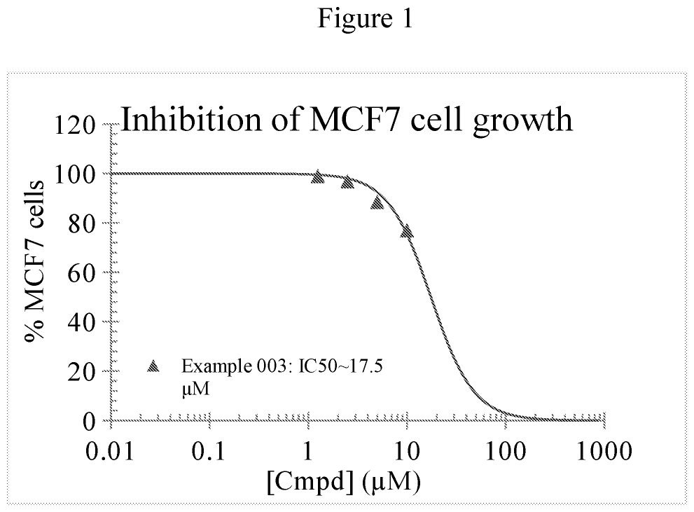

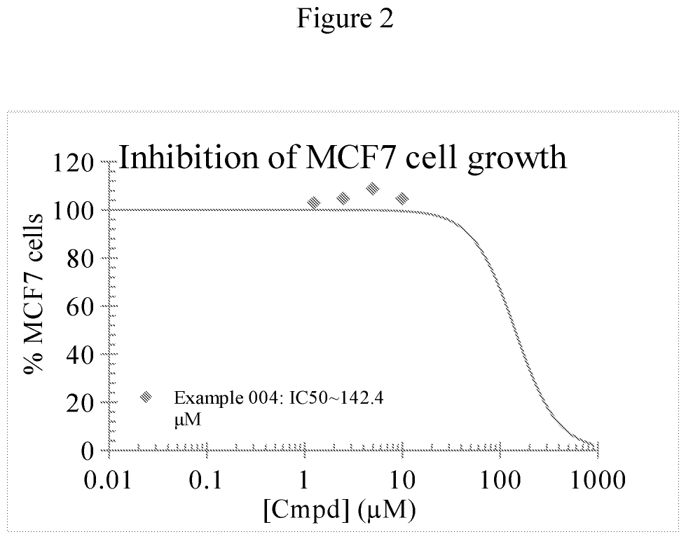

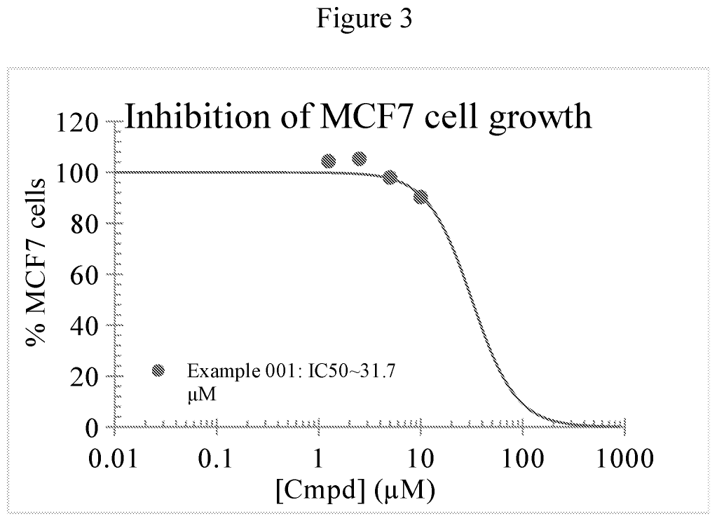

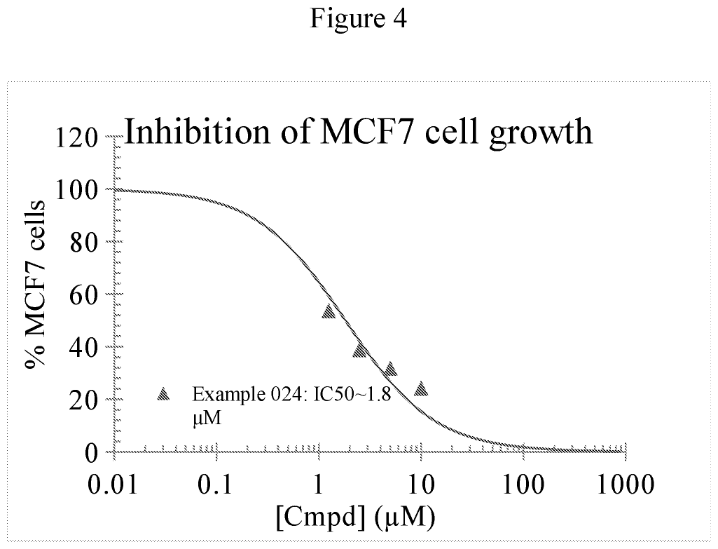

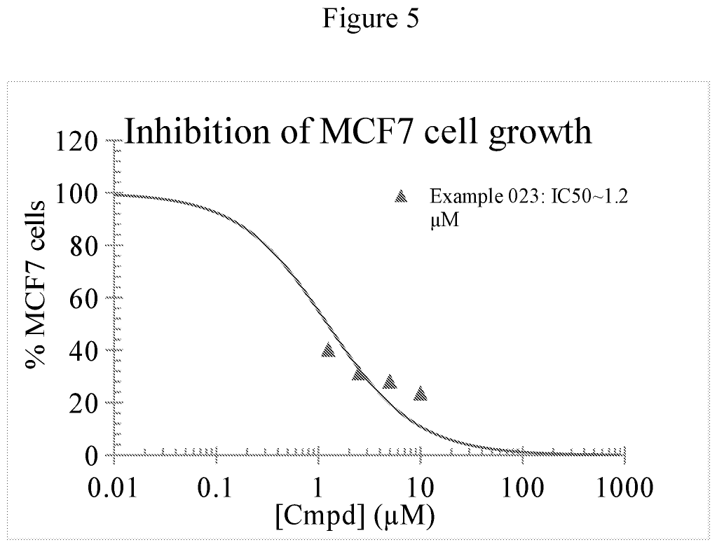

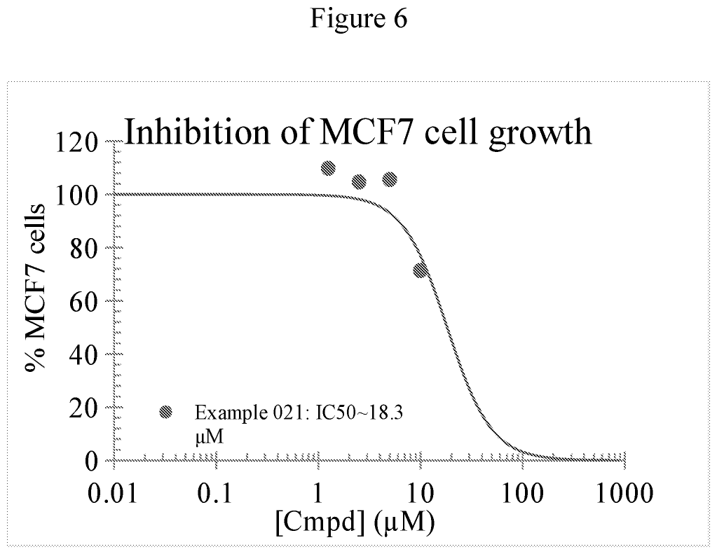

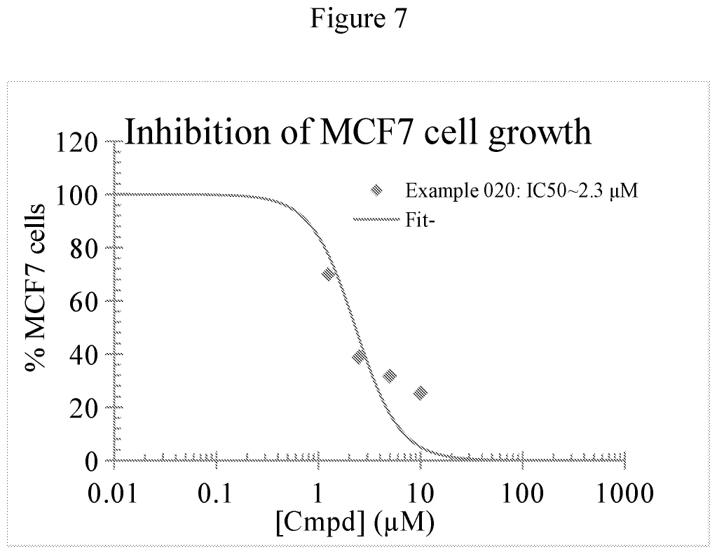

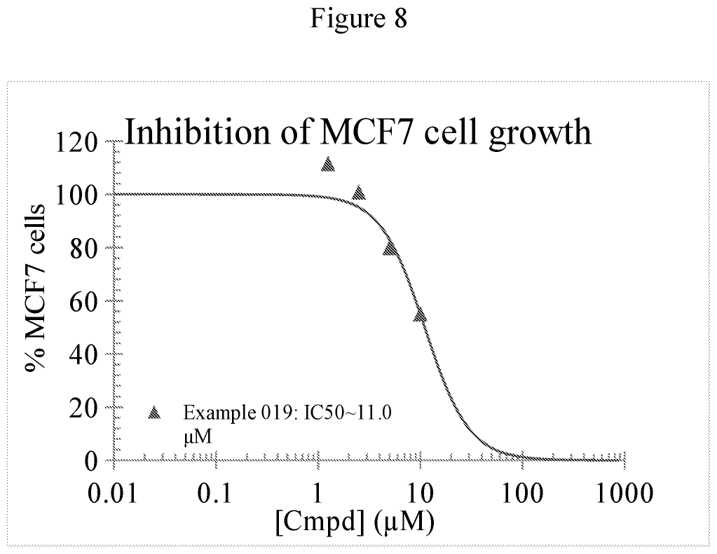

MCF7 cells were seeded in 96-well plates at 10% confluence 24 h prior to treatment. Compound was added to cells in full growth media containing 10% serum, using a 2-fold, 4-pt dilution scheme beginning from 10 μM (10, 5, 2.5, 1.25 μM). Plates were incubated for 24 h. After treatment, cells were washed, fixed with 4% PFA and stained with DAPI. Plates were imaged using a Cytation 3 plate reader and nuclear DAPI staining was used to quantify cell number. % Growth relative to vehicle-treated (DMSO) control wells was plotted using Excel (See FIGS. 1-8). IC50s were calculated by fitting the data points to the following standard equation:

Growth=1−[Cmpd]ⁿ/(IC50ⁿ+[Cmpd]ⁿ)

where [Cmpd] is the molar concentration of compound, the IC50 is the concentration where half-maximal effect is seen, and n is the Hill coefficient. The MCF7 Growth Inhibition plots of eight compounds of the invention are presented in FIGS. 1 through 8.

### MRC5 4d Toxicity Assay

MRC5 cells were seeded in 96-well plates 1-2 days prior to treatment to ensure 100% confluence. On the day of treatment, cells were washed once and growth media was replaced. Compound (in DMSO) was added to cells using the following dilution scheme: 25, 12.5, 4.16, 1.4, 0.46, 0.15, 0.05 μM. Test concentrations were assayed in duplicate and included 0 μM (DMSO) control wells. Plates were incubated for 4 days. After treatment, cells were washed, fixed with 4% PFA and stained with DAPI. Plates were imaged using a Cytation 3 plate reader and nuclear DAPI staining was used to quantify cell survival. Dose-response plots were generated using CDD Vault to calculate CC50s.

| | MCF7: GI50 | MRC5 4 d Tox: |
|---|---|---|
| Example | (μM) | CC50 (μM) |
| Example 001 | 31.7 | >25 |
| Example 003 | 17.5 | >25 |
| Example 004 | 142.3 | >25 |
| Example 019 | 11 | >25 |
| Example 020 | 2.3 | >25 |
| Example 021 | 18.3 | >25 |
| Example 022 | 18.3 | >25 |
| Example 023 | 1.2 | >25 |
| Example 024 | 1.8 | >25 |

### cMYC Degradation Assay

MDA-MB-231 cells were seeded in 6-well format in complete growth media containing 10% serum and incubated for 48 h to ensure 100% confluence. Compound was added to cells at 5 μM and 10 μM final concentrations for a treatment period of 72 hours. Cells were harvested in ice-cold IP buffer and lysates were cleared by centrifugation. Total protein concentration was determined for each sample by Bradford assay. Proteins were separated by 10% SDS-PAGE and blotted onto PVDF membranes. Membranes were blocked with CosmoBio PDVF blocking reagent for 1 hour, followed by overnight incubation with cMYC primary antibody (Abcam) at 4 C. After several washes, the membanes were probed with secondary antibody for 1 hour at room temperature. Fluorescent signal was acquired using an Odyssey CLx (LI-COR) and quantified using ImageJ software.

| | MDA-MB-231 | MDA-MB-231 | MDA-MB-231 | MDA-MB-231 | |
|---|---|---|---|---|---|
| | cMYC: Fold | cMYC: Fold | cMYC: Fold | cMYC: Fold | MDA-MB-231 |
| | Increase in | Decrease in | Increase in | Decrease in | cMYC |
| | High MW | Low MW Band | High MW | Low MW Band | qualitative |
| Example | Band at 5 μM | at 5 μM | Band at 10 μM | at 10 μM | degredation |
| 001 | 0.84 | 0.98 | 1.7 | 1.2 | no degradation |
| 007 | 0.9 | 0.63 | 1.3 | 0.8 | no degradation |
| 011 | 1.53 | 0.31 | 2.6 | 0.5 | partial |
| | | | | | degradation |
| 019 | 1.18 | 0.62 | 3.2 | 0.9 | partial |
| | | | | | degradation |
| 023 | 0.22 | 0.04 | 0.2 | 0.0 | total |
| | | | | | degradation |

### Assessing Antiviral Activity Against Human Cytomegalovirus (HCMV)

To assess their antiviral activity, some compounds were tested against human cytomegalovirus (HCMV) in vitro. Human MRC5 cells were grown to confluency (˜1.0×10\hat{ }4 cells/well) in 96-well plate format in Dulbecco's Modified Eagle Medium (DMEM) supplemented with 10% fetal bovine serum (FBS) 2 mM L-glutamine, 0.1 mM non-essential amino acids, 10 mM HEPES, and 100 U/ml each of penicillin and streptomycin and infected with an HCMV variant expressing mCherry tagged pUL99 (the product of late viral UL99 gene) at a multiplicity of 0.01 infectious unit (IU) per cell. Assays were performed in triplicate. One hour later, medium of the cells was replaced with fresh medium containing the indicated compounds at 25, 12.5, 6.25, 3.13, 1.56, 0.78, 0.39 μM or the carrier in which the compounds are dissolved (DMSO). Final concentration of DMSO was 0.5% in each treatment. Virus yield in the culture was determined at 7 days post infection by quantification of fluorescent (mCherry positive) cells in each well by fluorescent microscopy. Results were plotted using CDD Vault (CDD Vault was developed by Collaborative Drug Discovery, Inc., 1633 Bayshore Hwy, Suite 342, Burlingame, CA 94010) in order to calculate IC50s and IC90s. Results of compounds tested with this assay are provided in Table 1.

| | MRC5 TOX | | | |
|---|---|---|---|---|
| | HCMV: Avg | HCMV: Avg | 7 d: Avg | Selectivity |
| | IC50 | IC90 | CC50 | Index: |
| Example | (μM) | (μM) | (μM) | SI50 |
| Example 001 | 1.459 | 8.72 | >10.400 | >7.1 |
| Example 002 | 2.59 | 7.58 | >50.000 | >19.3 |
| Example 003 | 5.771 | >6.77 | >6.770 | >1.2 |
| Example 004 | 5.587 | >25.0 | >15.800 | >2.8 |
| Example 005 | 1.431 | 2.92 | >10.400 | >7.3 |
| Example 006 | 3.366 | >50.0 | 43.351 | 12.9 |
| Example 007 | 1.188 | >2.89 | >2.890 | >2.4 |
| Example 008 | 1.095 | 3.79 | >14.486 | >13.2 |
| Example 009 | 2.045 | 7.43 | >50.000 | >24.4 |
| Example 010 | 2.445 | 7.97 | 16.858 | 6.9 |
| Example 011 | 1.476 | >10.4 | >10.400 | >7.0 |
| Example 012 | 2.128 | >4.24 | >50.000 | >23.5 |
| Example 013 | 4.274 | 9.25 | >50.000 | >11.7 |
| Example 014 | 3.503 | >6.77 | >6.770 | >1.9 |
| Example 015 | 2.508 | 5.59 | 44.436 | 17.7 |
| Example 016 | 5.25 | >6.77 | >15.800 | >3.0 |
| Example 017 | 6.458 | 47.5 | 47.343 | 7.3 |
| Example 018 | 16.137 | 17.7 | >50.000 | >3.1 |
| Example 019 | 0.791 | >6.61 | >6.770 | >8.6 |
| Example 020 | 1.244 | 4.7 | 3.631 | 2.9 |
| Example 021 | 1.173 | >4.43 | >2.890 | >2.5 |
| Example 022 | 0.536 | >4.43 | >4.430 | >8.3 |
| Example 023 | 0.874 | 2.9 | >13.574 | >15.5 |
| Example 024 | 1.143 | 5.31 | 21.4 | 18.7 |

### Assessing Antiviral Activity Against Coronaviruses

To assess their antiviral activity, some compounds will be tested against Human Coronavirus OC43 (HcoV-0c43) in vitro. HCoV-OC43 infected and uninfected MRC5 cells will be treated with compound at a range of concentrations for a period of 6 days. OC43 infection of MRC5 cells at a low multiplicity will result in >50% cytopathic effect (CPE), or release of adherent cells by the end of the sixth day. Cytoprotection will be measured by comparing cell adherence (indicated by nuclear DAPI staining) in test wells to uninfected, vehicle treated wells (Uninfected Control, UC) and infected, vehicle treated wells (Virus Control, VC). MRC5 cells will be seeded in 96-well clear, flat-bottom TPP plates and incubated at 37° in DMEM 10% FBS for 2-3 days until the cells have reached >90% confluence. On the day of the infection, DMEM 10% FBS and serum-free DMEM media will be warmed to 37 C in a waterbath. DMEM 2% FBS will be made by mixing 1 part DMEM 10% FBS with 4 parts serum-free DMEM. Working with plates one at a time, aspirate PBS and refeed cells with DMEM 2% FBS by addition of 50 uL (for OC43 infection plates) or 100 uL (for tox plates). Promptly return plates to 37 C incubator. An appropriate volume of concentrated virus stock will be diluted in DMEM 2% FBS to achieve a MOI=0.05 IU/cell. Using a multichannel pipette, 50 uL of virus-containing media will be added across each of the infection plates and the plates promptly returned to the 37° C. incubator. At 6 dpi, plates will be fixed with 4% PFA and stained with DAPI (lug/ml). Cell counts from each stained well will be obtained and IC50s for the compounds will be calculated.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A composition comprising a compound of Formula I: 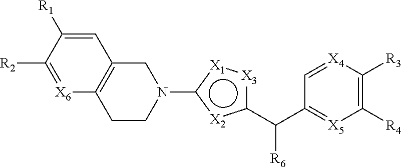
wherein:
one of X1, X2 and X3 is —S— or —O—, and
X1, when not —S— or —O—, is —N—,
X2, when not —S— or —O—, is —N—, and
X3, when not —S— or —O—, is —N— or —C(R5)—, wherein R5 is selected from the group consisting of H, methyl, ethyl, n-propyl, isopropyl, n-butyl, CF₃, CH₂CF₃ and halo;
X4 is selected from —C(R13)— and —N—, wherein R13 is selected from the group consisting of H, methyl, ethyl, i-propyl or n-propyl;
X5 is selected from —CH— and —N—;
X6 is selected from —C(R14)— and —N—, wherein R14 is H or halo;
one of R1 and R2 is H and the other is 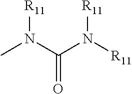
wherein R11 is independently selected, in each case, from H, methyl, ethyl, n-propyl & i-propyl;
R3 and R4 are independently selected from H, halo, —C≡CH, —C≡N, —OH, —OCF₃, —OCHF₂, C₁₋₄ straight or branched alkoxy optionally substituted with a 3- or 4-membered cycloalkyl, —SO₂(C₁₋₆ alkyl), —N(CH₃)₂, —C(O)NH₂, —NHSO₂R7, —C(O)NR7R8, and a ring structure comprising a 5- or 6-membered aryl or a 3-, 4-, 5-, or 6-membered cylcloalkyl or a 3-, 4-, 5-, or 6-membered cycloalkoxy;
wherein:
each 5-, or 6-membered aryl, 4-, 5-, or 6-membered cycloalkyl or 4-, 5-, or 6-membered cycloalkoxy has 0 to 3 ring heteroatoms and each 3-membered cycloalkyl or cycloalkoxy has 0 to 1 heteroatoms,
each heteroatom is independently selected from N, O and S and
each aryl, cycloalkyl or cycloalkoxy is substituted with 0 to 2 groups independently selected from:
═O, halo, C₁₋₆ straight or branched alkyl optionally substituted with —OR12 or —NR7R8, C₁₋₆ straight or branched alkoxy optionally substituted with —NR7R8 or —OR12, —C(O)—C₁₋₆ alkyl and —C(O)O—C₁₋₆ alkyl bonded to a carbon ring atom;
O, C₁₋₆ straight or branched alkyl optionally substituted with —OR12 or —NR7R8, C₁₋₆ straight or branched alkoxy optionally substituted with —NR7R8 or —OR12, —C(O)—C₁₋₆ alkyl and —C(O)O—C₁₋₆ alkyl bonded to a nitrogen ring atom; and
═O and (═O)₂ bonded to a sulfur ring atom;
provided that:
at least one of R3 and R4 is selected from the group consisting of: H, halo, —C≡CH, —C≡N, —OH, —OCF₃, —OCHF₂, C₁₋₄ straight or branched alkoxy optionally substituted with cyclopropyl or cyclobutyl, —SO₂(C₁₋₆ alkyl), —N(CH₃)₂, —C(O)NH₂, —NHSO₂R7, and —C(O)NR7R8,
when R3 or R4 is an aryl group, said aryl group is not substituted with ═O, and R3 and R4 are not both H;
R6 is selected from the group consisting of H, methyl, ethyl, n-propyl, isopropyl, n-butyl, CF₃, CH₂CF₃, halo, cyclopropylmethyl and C₁₋₄ alkoxy;
R7 and R8 are independently selected, in each instance, from H, C₁₋₆ straight or branched alkyl, C₃₋₆ cycloalkyl, cyclopropylmethyl and cyclobutylmethyl; and
R12 is independently selected, in each instance, from H and C₁₋₄ straight or branched alkyl;
or a pharmaceutically acceptable salt or solvate thereof.

2. The composition of claim 1, wherein one of R3 or R4 is:
a C₁₋₄ straight or branched alkoxy optionally substituted with a 3- or 4-membered cycloalkyl, or
a ring structure comprising a 5- or 6-membered aryl or a 3-, 4-, 5-, or 6-membered cylcloalkyl or a 3-, 4-, 5-, or 6-membered cycloalkoxy;
wherein:
each 5-, or 6-membered aryl, 4-, 5-, or 6-membered cycloalkyl or 4-, 5-, or 6-membered cycloalkoxy has 0 to 3 ring heteroatoms and each 3-membered cycloalkyl or cycloalkoxy has 0 to 1 heteroatoms;
each heteroatom is independently selected from N, O and S; and
each aryl, cycloalkyl or cycloalkoxy is substituted with 0 to 2 groups independently selected from:
═O, halo, C₁₋₆ straight or branched alkyl optionally substituted with —OR12 or —NR7R8, C₁₋₆ straight or branched alkoxy optionally substituted with —NR7R8 or —OR12, —C(O)—C₁₋₆ alkyl and —C(O)O—C₁₋₆ alkyl bonded to a carbon ring atom;
O, C₁₋₆ straight or branched alkyl optionally substituted with —OR12 or —NR7R8, C₁₋₆ straight or branched alkoxy optionally substituted with —NR7R8 or —OR12, —C(O)—C₁₋₆ alkyl and —C(O)O—C₁₋₆ alkyl bonded to a nitrogen ring atom; and
═O and (═O)₂ bonded to a sulfur ring atom.

3. The composition of claim 2, wherein X2 is —S— or —O—.

4. The composition of claim 3, wherein X3 is —C(R5)—.

5. The composition of claim 3, wherein X2 is —S—.

6. The composition of claim 5, wherein X3 is —C(R5)—.

7. The composition of claim 1, wherein:
R3 is selected from the group consisting of: 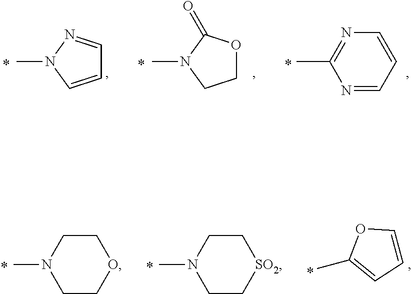
—SO₂(C₁₋₆ alkyl) and C₁₋₄ straight or branched alkoxy, wherein the alkoxy is optionally substituted with a 3- or 4-membered cycloalkyl,
wherein:
the 3- or 4-membered cycloalkyl has 0 to 1 heteroatoms independently selected from N, O and S; and
the 3- or 4-membered cycloalkyl is substituted with 0 to 2 groups independently selected from:
═O, halo, C₁₋₄ straight or branched alkyl optionally substituted with —OR12 or —NR7R8, C₁₋₄ straight or branched alkoxy optionally substituted with —NR7R8 or —OR12, —C(O)—C₁₋₄ alkyl and —C(O)O—C₁₋₄ alkyl bonded to a carbon ring atom; and
C₁₋₆ straight or branched alkyl optionally substituted with —OR12 or —NR7R8, C₁₋₆ straight or branched alkoxy optionally substituted with —NR7R8 or —OR12, —C(O)-C₁₋₄ alkyl and —C(O)O—C₁₋₄ alkyl bonded to a nitrogen ring atom.

8. The composition of claim 7, wherein the composition is selected from the group consisting of: 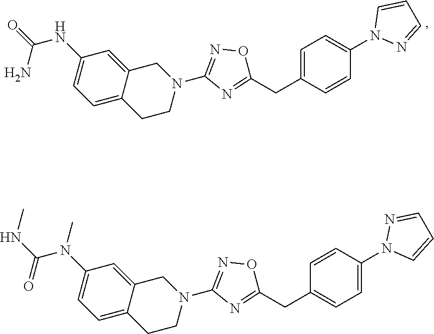 [SMILES: NC(=O)Nc1ccc2c(c1)CN(c1noc(Cc3ccc(-n4cccn4)cc3)n1)CC2 ; CNC(=O)N(C)c1ccc2c(c1)CN(c1noc(Cc3ccc(-n4cccn4)cc3)n1)CC2]
and a pharmaceutically acceptable solvate thereof.

9. The composition of claim 7, wherein wherein X2 is —S— or —O—.

10. The composition of claim 9, comprising 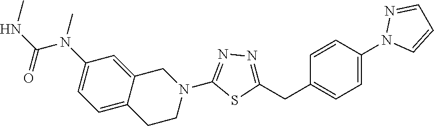 [SMILES: CNC(=O)N(C)c1ccc2c(c1)CN(c1nnc(Cc3ccc(-n4cccn4)cc3)s1)CC2]
or a phramaceutically acceptable solvate thereof.

11. The composition of claim 9, wherein X3 is —C(R5)—.

12. The composition of claim 9, wherein X2 is —S—.

13. The composition of claim 12, wherein X3 is —C(R5)—.

14. The composition of claim 13, selected from the group consisting of: 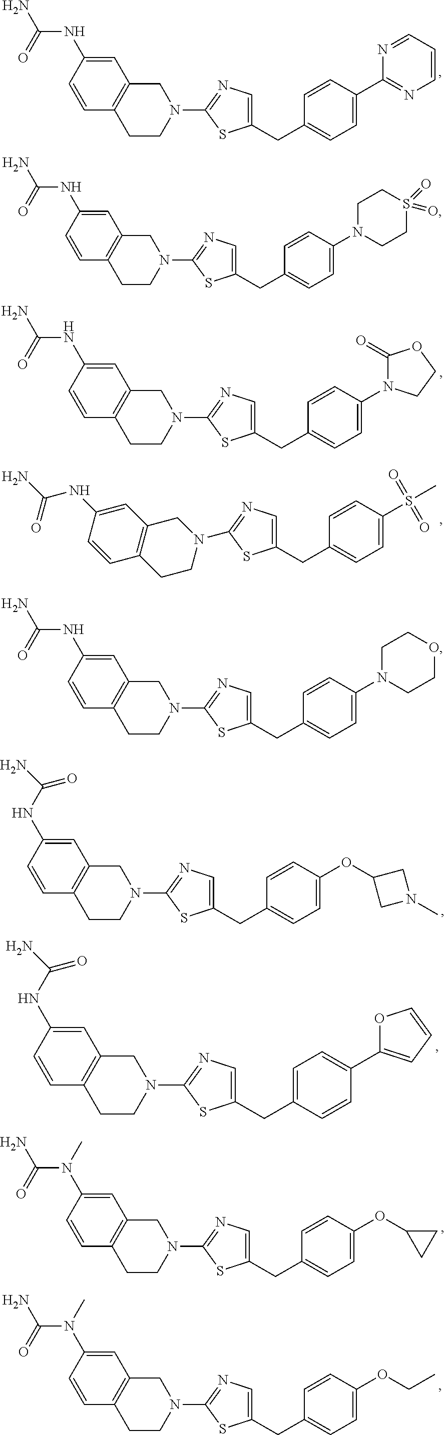 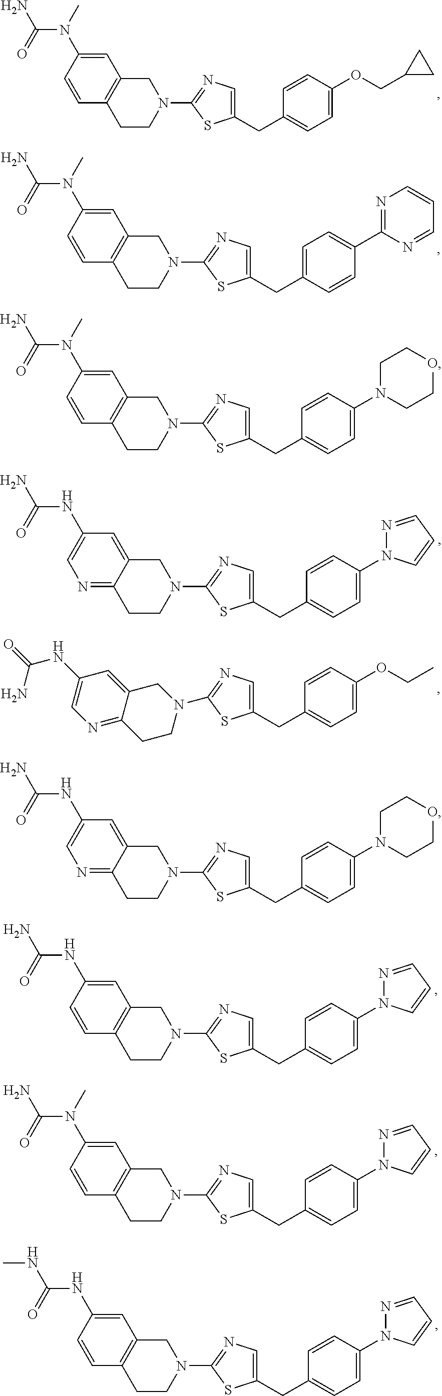 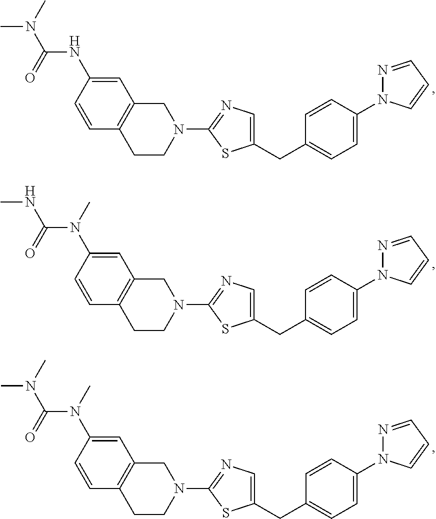 [SMILES: CN(C)C(=O)Nc1ccc2c(c1)CN(c1ncc(Cc3ccc(-n4cccn4)cc3)s1)CC2 ; CNC(=O)N(C)c1ccc2c(c1)CN(c1ncc(Cc3ccc(-n4cccn4)cc3)s1)CC2 ; CN(C)C(=O)N(C)c1ccc2c(c1)CN(c1ncc(Cc3ccc(-n4cccn4)cc3)s1)CC2]
and a pharmaceutically acceptable salt or solvate thereof.

15. The composition of claim 1, wherein:
R4 is selected from the group consisting of: 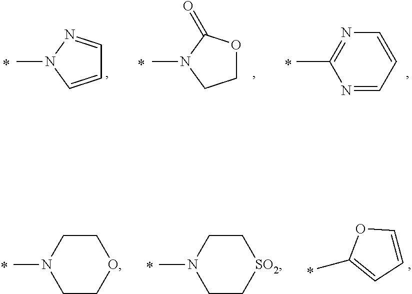
—SO₂(C₁₋₆ alkyl) and C₁₋₄ straight or branched alkoxy, wherein the alkoxy is optionally substituted with a 3- or 4-membered cycloalkyl,
wherein:
the 3- or 4-membered cycloalkyl has 0 to 1 heteroatoms independently selected from N, O and S; and
the 3- or 4-membered cycloalkyl is substituted with 0 to 2 groups independently selected from:
═O, halo, C₁₋₄ straight or branched alkyl optionally substituted with —OR12 or —NR7R8, C₁₋₄ straight or branched alkoxy optionally substituted with —NR7R8 or —OR12, —C(O)—C₁₋₄ alkyl and —C(O)O—C₁₋₄ alkyl bonded to a carbon ring atom; and
C₁₋₆ straight or branched alkyl optionally substituted with —OR12 or —NR7R8, C₁₋₆ straight or branched alkoxy optionally substituted with —NR7R8 or —OR12, —C(O)—C₁₋₄ alkyl and —C(O)O—C₁₋₄ alkyl bonded to a nitrogen ring atom.

16. (canceled)

17. A method of treating cancer in a patient in need of treatment comprising administering to said patient a therapeutically effective amount of a composition of claim 1.

18. The method of claim 17, wherein said cancer is breast cancer.

19. A method for treating or preventing a viral infection in a subject, the method comprising administering to the subject a therapeutically effective amount of a composition claim 1.

20. (canceled)

21. The method of of claim 17, comprising administering to said patient a therapeutically effective amount of a composition of claim 2.

22. The method of claim 17, comprising administering to said patient a therapeutically effective amount of a composition of claim 7.
